(19) United States
(12) Patent Application Publication
(10) Pub. No.: US 2005/0149999 A1
(43) Pub. Date: Jul. 7, 2005

(54) Methods for cloning mammals using remodeling factors

(75) Inventors: Gregory H Leno, Madison, WI (US); Kenneth Eilertsen, Baton Rouge, LA (US); Jeffrey M Bethauser, Windsor, WI (US); Erik J Forsberg, Oregon, WI (US)

Correspondence Address: FOLEY & LARDNER, P.O. BOX 80278, SAN DIEGO, CA, 92138-0278, US

(73) Assignee: Infigen Inc.

(21) Appl. No.: 10/480,515

(22) PCT Filed: Jun. 14, 2002
(86) PCT No.: PCT/US02/19103

Related U.S. Application Data
(60) Provisional application No. 60/298,574, filed on Jun. 14, 2001.

(51) Int. Cl. A01K 67/027

(52) U.S. Cl. 800/15; 800/16; 800/17; 800/21

(57) ABSTRACT

Methods and compositions are provided for remodeling nuclear donor material used in nuclear transfer procedures. By exposing donor chromatin to one or more exogenous remodeling factors, the limited ability of mammalian oocytes to remodel the chromatin of differentiated cells, including fetal and live-born somatic cells, can be increased, resulting in dramatically improved cloning efficiencies.

### RELATED APPLICATIONS

This application is related to U.S. Provisional Application Ser. No. 60/298,574 entitled “Methods for Cloning Mammals Using Remodeling Factors,” filed Jun. 14, 2001. That application is incorporated herein by reference as if fully set forth in this application.

### FIELD OF THE INVENTION

The present invention relates to methods of cloning mammals.

### BACKGROUND OF THE INVENTION

The following discussion of the background of the invention is provided to aid the reader in understanding the invention and is not admitted to describe or constitute prior art to the present invention.

Over the past two decades, researchers have been developing methods for cloning mammalian animals, with notable recent success. The reported methods typically include the steps of (1) isolating a cell, often an embryonic cell, but more recently fetal and adult cells as well; (2) inserting the cell or nucleus isolated from the cell into an enucleated recipient cell (e.g., an NT oocyte as defined herein, the nucleus of which was previously extracted), (3) activating the oocyte, and (4) allowing the embryo to mature in vivo. See, e.g., U.S. Pat. No. 4,664,097, “Nuclear Transplantation in the Mammalian Embryo by Microsurgery and Cell Fusion,” issued May 12, 1987, McGrath & Solter; U.S. Pat. No. 4,994,384 (Prather et al.); U.S. Pat. No. 5,057,420 Massey et al.); U.S. Pat. No. 6,107,543; U.S. Pat. No. 6,011,197; Proc. Nat'l. Acad. Sci. USA 96: 14984-14989 (1999); Nature Genetics 22: 127-128 (1999); Cell & Dev. Biol. 10: 253-258 (1999); Nature Biotechnology 17: 456-461 (1999); Science 289: 1188-1190 (2000); Nature Biotechnol. 18: 1055-1059 (2000); and Nature 407: 86-90 (2000); each of which is incorporated herein by reference in its entirety, including all figures, tables, and drawings.

Successful development of any cloned embryo is believed to involve the “reprogramming” of the somatic nucleus by the egg cytoplasm. Reprogramming involves reversing the genetic programming of the differentiated somatic cell to create a totipotent nucleus. Chromatin structure partly determines the cell's epigenetic memory, which regulates the pattern of gene expression in its descendants. Thus, the regulated change in structure or “remodeling” of somatic chromatin by the egg may reverse this pattern of expression, and as such, facilitate development.

Successful cloning demonstrates that the unfertilized egg has the potential to direct the complete reprogramming of the somatic nucleus. However, the relative inefficiency of the process also suggests that important activities can be limiting in nuclear transfer events (Gurdon and Colman, Nature, 402(6763): p. 743-6, 1999). While specific features of donor nuclei certainly contribute to this inefficiency, in theory, virtually all somatic nuclei may have the potential to become totipotent for development if they are correctly and completely reprogrammed. Thus, it may be that it is primarily the limited reprogramming capacity of the egg that is responsible for most cloning failures. This limitation is undoubtedly due to a number of factors, as the egg has evolved to program a sperm nucleus at fertilization and not to reprogram a somatic nucleus following nuclear transfer. However, while many factors may be necessary for complete reprogramming, it is possible that reprogramming can be achieved with only a few factors. See, e.g., Kikyo and Wolffe, J. Cell Sci. 113: 11-20, 2000. If so, then supplementing the egg with these critical factors, or treating somatic nuclei with these factors prior to nuclear transfer, may result in improved development and increased cloning success.

Thus, despite the recent progress in cloning mammalian animals, there remains a great need in the art for methods and materials that increase cloning efficiencies.

### SUMMARY OF THE INVENTION

The present invention provides methods for cloning mammals by nuclear transfer. As described herein, exposing an oocyte and/or a somatic cell or nucleus to remodeling factors prior to their use in nuclear transfer procedures can increase the efficiencies of cellular reprogramming. Moreover, by careful selection of such remodeling factors, it can be possible to achieve these increased efficiencies utilizing only one or a small number of remodeling factors. Preferred remodeling factors include, but are not limited to, nucleoplasmin, cyclin A-dependent kinase(s), protein kinases, or a combination of these.

The present invention therefore provides, in a first aspect, methods and compositions for preparing a mammalian embryo by nuclear transfer. The methods may comprise transferring a mammalian cell, or the nucleus thereof, into an enucleated mammalian oocyte, introducing into the mammalian oocyte one or more remodeling factors prior to, subsequent to, or simultaneous with the transferring step, and activating the mammalian oocyte to provide an embryo.

For purposes of clarity, the mammalian oocyte that is to receive or has received the nuclear donor cell or nucleus is referred to hereinafter as an “NT oocyte.” This is to distinguish such oocytes from those that are used as the source of reprogramming factors and extracts. This designation is purely for convenience, and does not denote that the NT oocyte has received a donor cell or nucleus at the time to which it is referred.

In certain embodiments, the methods may comprise preparing an embryo by the methods of the present invention, and transferring the embryo, or a re-cloned embryo thereof, into the uterus of a host mammal so as to produce a fetus that undergoes full development and parturition. “Re-cloning” is described hereinafter.

The term “mammalian” as used herein refers to any animal of the class Mammalia. Preferably, a mammal is a placental, a monotreme and a marsupial. Most preferably, a mammal is a canid, felid, murid, leporid, ursid, mustelid, ungulate, ovid, suid, equid, bovid, caprid, cervid, and a human or non-human primate. These terms are defined hereinafter.

In preferred embodiments, the mammal may be a bovine, the mammalian NT oocyte may be a bovine oocyte, and/or the mammalian cell may be a bovine cell; the mammal may be a porcine, the mammalian NT oocyte may be a porcine oocyte, and/or the mammalian cells may be porcine cells; and the mammal may be an ovine, the mammalian NT oocyte may be an ovine oocyte, and/or the mammalian cells may be ovine cells.

The one or more remodeling factors may be obtained from cells, such as oocytes and eggs, at any stage of maturation and/or development. Thus, the remodeling factors of the instant invention may be obtained before and/or after activation of the source cells. In addition, remodeling factors may also be obtained from cells from multiple maturation and/or developmental stages and pooled.

While any species may serve as the source of these remodeling factors, amphibian oocytes and eggs, and particularly Xenopus oocytes, Xenopus eggs, and activated Xenopus eggs, are a particularly rich source of these remodeling factors.

The term “oocyte” as used herein with reference to amphibian cells refers to a female germ cell arrested in G2/prophase of meiosis I.

The term “egg” as used herein with reference to amphibian cells refers to a c0 female germ cell arrested in metaphase of meiosis II.

The term “activated egg” as used herein with reference to amphibian cells refers to a female germ cell that is beyond the “egg” stage due to release from metaphase arrest and progression into interphase.

Each of the previous three definitions are known to the skilled artisan. See, e.g., Leno, Methods in Cell Biology 53: 497-515, 1998.

Extracts of such cells may be used without fractionation, as these extracts contain the remodeling factors; but in certain embodiments the remodeling factors may be purified factors such as nucleoplasmin, cyclin A-dependent kinase, ATP-dependent chromatin remodeling complexes, or a combination thereof. Remodeling factors may also be obtained by recombinant methods. For example, insect cells may be transformed to produce Xenopus nucleoplasmin, which may be used in the methods described herein. Similarly, mRNA obtained from, for example, Xenopus cells may be translated in vitro to produce Xenopus remodeling factors. Purification in this context does not indicate absolute purity; only that the relative amount of a preferred compound has been enriched.

The term “remodeling factor” as used herein refers to any substance that alters the structure and/or composition of chromatin, known as “chromatin restructuring.” Remodeling factors include, but are not limited to, ATP-dependent remodeling factors (e.g., SWI/SNF, ISWI, and ISWI homologs from yeast and Xenopus; see, e.g., Genes & Development 15: 619-26, 2001; and cyclin-dependent kinases; see, e.g., Hua et al., J. Cell. Biol. 137: 183-192, 1997; Findeisen et al., Eur. J. Biochem. 264: 415-26, 1999); non-ATP-dependent remodeling factors (e.g., nucleoplasmin and polyanionic molecules such as polyglutamic acid (Philpot and Leno, Cell 69: 759-67, 1992; Dean, Dev. Biol. 99: 210-216, 1983); and chromatin components that can replace their counterparts that are preexisting in chromatin (e.g., histone H1ₒₒ or H1_{embryonic} may replace histone H1_{somatic}). Whole cell extracts, whether unpurified or purified, that precipitate chromatin restructuring can also be referred to as remodeling factors.

Preferably, the one or more remodeling factors may be introduced into a cell, such as an NT oocyte or a nuclear donor cell, by microinjection (for example using a piezo drill), by delivery in liposomes (e.g., BioPORTER, Gene Therapy Systems, San Diego, Calif.), by transient permeabilization of the recipient cell (e.g. by streptolysin 0 or digitonin treatment), by electroporation, or by any other methods for introducing materials into cells that are known to the artisan.

The skilled artisan will recognize that the use of an NT oocyte in these procedures provides a reservoir for exposing nuclear donor material to levels of remodeling factors sufficient to cause successful remodeling of the donor chromatin. Thus, any small chamber may be used as a replacement for the NT oocyte. For example, an enucleated cell of any type (e.g., an enucleated zygote, an enucleated blastomere, etc.) may receive the nuclear donor material and remodeling factor(s). Alternatively, any chamber of approximately the size of a cell (a liposome, chromatin encapsulated by an artificial membrane, etc.) may also be used as a reprogramming chamber. Thus, while the specification discusses the use of NT oocytes, other such reprogramming chambers are within the scope of the invention. In particular, any cultured cell may be considered an appropriate reprogramming chamber; that is, the nucleus may be exposed within the cell to reprogramming factors, and then that cell itself may be treated as one would a nuclear transfer-derived embryo (e.g., to transfer to a recipient animal for development into a fetus or live-born animal, or as a source of cultured cells such as stem cells or stem cell-like cells).

Remodeling factor(s) can be introduced into the nuclear transfer procedure at various points. For example, remodeling factor(s) may be introduced into an NT oocyte prior to, subsequent to, or simultaneously with the transfer of nuclear donor material into the NT oocyte. Similarly, remodeling factors can be introduced into an NT oocyte before or after enucleation of the NT oocyte, before, during, or after maturation of an NT oocyte, or before, during, or after activation of the NT oocyte. In preferred embodiments, remodeling factors are introduced between 20 hours before activation and the time of activation, more preferably between 10 hours before activation and the time of activation.

Remodeling factor(s) can also be introduced into the nuclear transfer procedure following the generation of a nuclear transfer-derived embryo. For example, remodeling factor(s) may be introduced into a developing embryo in culture.

The mammalian cell used as a source of nuclear donor material may be any mammalian cell, but is preferably an embryonic cell, a fetal cell, a fetal fibroblast cell, an adult cell, a somatic cell, a primordial germ cell, a genital ridge cell, a fibroblast cell, a cumulus cell, an amniotic cell, an embryonic germ cell, an embryonic stem cell, an ovarian follicular cell, a hepatic cell, an epidermal cell, an epithelial cell, a hematopoietic cell, keratinocyte, a renal cell, a lymphocyte, a melanocyte, a muscle cell, a myeloid cell, a neuronal cell, an osteoblast, a mysenchymal cell, a mesodermal cell, an adherent cell, a cell isolated from an asynchronous population of cells, a cell isolated from a synchronous population of cells where the synchronous population is not arrested in the G0 stage of the cell cycle, a transgenic embryonic cell, a transgenic fetal cell, a transgenic adult cell, a transgenic somatic cell, a transgenic primordial germ cell, a transgenic fibroblast cell, a transgenic cumulus cell or a transgenic amniotic cell.

In particularly preferred embodiments, a nuclear donor cell is a transgenic cell. The term “transgenic” as used herein in reference to cells refers to a cell whose genome has been altered using recombinant DNA techniques. In preferred embodiments, a transgenic cell comprises one or more exogenous DNA sequences in its genome. In other preferred embodiments, a transgenic cell comprises a genome in which one or more endogenous genes have been deleted, duplicated, activated, or modified. In particularly preferred embodiments, a transgenic cell comprises a genome having both one or more exogenous DNA sequences, and one or more endogenous genes that have been deleted, duplicated, activated, or modified.

In another aspect, the methods of the present invention for preparing a mammalian embryo by nuclear transfer may comprise transferring a mammalian cell, or the nucleus thereof, into an enucleated mammalian NT oocyte, introducing into the mammalian NT oocyte a cytoplasmic extract obtained from one or more cells, preferably amphibian cells (e.g., Xenopus oocytes, Xenopus eggs, and activated Xenopus eggs), prior to, subsequent to, or simultaneous with the transferring step, and activating the mammalian NT oocyte to provide the embryo.

In certain embodiments, the methods may comprise preparing an embryo according to the present invention, and transferring the embryo or a re-cloned embryo thereof into the uterus of a host mammal so as to produce a fetus that undergoes full development and parturition.

In yet another aspect, the present invention provides methods for preparing a mammalian embryo by nuclear transfer comprising contacting a mammalian cell, or a nucleus thereof, with one or more remodeling factors, transferring the mammalian cell, or the nucleus thereof, into an enucleated mammalian egg, and activating the egg to provide the embryo.

In various embodiments, the plasma membrane of the mammalian cell may be permeabilized and/or the nuclear membrane of the mammalian cell nucleus may be permeabilized by methods known to the skilled artisan, in order to permit the remodeling factor(s) to access the interior of the cell and/or nucleus. For example, in preferred embodiments, the plasma membrane of the mammalian cell may be permeabilized by exposure to streptolysin-O and/or digitonin prior to contacting the mammalian cell with the remodeling factors, and/or the nuclear membrane of the mammalian cell nucleus may be permeabilized by homogenization.

In addition to methods in which remodeling factors are introduced into mammalian cell nuclei by permeabilization of the nuclear membrane, in certain embodiments remodeling factors may also be introduced into a mammalian cell nucleus through the use of nuclear localization signals, or by using remodeling factors that are sufficiently small to diffuse through the nuclear pore complexes present in the nuclear membrane.

In another aspect the present invention provides methods for preparing a mammalian embryo by nuclear transfer comprising contacting a mammalian cell, or a nucleus thereof, with a cytoplasmic extract obtained from one or more cells such as Xenopus oocytes, Xenopus eggs, and activated Xenopus eggs, transferring the mammalian cell, or the nucleus thereof, into an enucleated mammalian NT oocyte, and activating the mammalian NT oocyte to provide the embryo.

The term “nuclear transfer” as used herein refers to introducing a full complement of nuclear DNA from one cell to an enucleated cell. Nuclear transfer methods are well known to a person of ordinary skill in the art. See, e.g., U.S. Pat. No. 4,664,097, “Nuclear Transplantation in the Mammalian Embryo by Microsurgery and Cell Fusion,” issued May 12, 1987, McGrath & Solter; U.S. Pat. No. 4,994,384 (Prather et al.); U.S. Pat. No. 5,057,420 (Massey et al.); U.S. Pat. No. 6,107,543; U.S. Pat. No. 6,011,197; Proc. Nat'l. Acad. Sci. USA 96: 14984-14989 (1999); Nature Genetics 22: 127-128 (1999); Cell & Dev. Diol 10: 253-258 (1999); Nature Biotechnology 17: 456-461 (1999); Science 289: 1188-1190(2000); Nature Biotechnol. 18: 1055-1059 (2000); and Nature 407: 86-90 (2000); each of which is incorporated herein by reference in its entirety, including all figures, tables, and drawings. Exemplary embodiments define a nuclear transfer technique that provide for efficient production of totipotent mammalian embryos.

The term “enucleated oocyte” as used herein refers to an oocyte which has had part of its contents removed. As discussed above, such an oocyte is also referred to herein as an “NT oocyte,” to distinguish these oocytes from cells that are the source of remodeling factors. Typically a needle can be placed into an oocyte and the nucleus can be aspirated into the inner space of the needle. The needle can be removed from the oocyte without rupturing the plasma membrane. This enucleation technique is well known to a person of ordinary skill in the art. See, U.S. Pat. No. 4,994,384; U.S. Pat. No. 5,057,420; and Willadsen, 1986, Nature 320: 63-65. An enucleated oocyte can be prepared from a young or an aged oocyte. Definitions of “young oocyte” and aged oocyte” are provided herein. Nuclear transfer may be accomplished by combining one nuclear donor and more than one enucleated oocyte. In addition, nuclear transfer may be accomplished by combining one nuclear donor, one or more enucleated oocytes, and the cytoplasm of one or more enucleated oocytes.

The term “injection” as used herein in reference to nuclear transfer methods, refers to the perforation of the NT oocyte with a needle, an insertion of the nuclear donor in the needle into the NT oocyte. In preferred embodiments, the nuclear donor may be injected into the cytoplasm of the NT oocyte or in the perivitelline space of the NT oocyte. This direct injection approach is well known to a person of ordinary skill in the art, as indicated by the publications already incorporated herein in reference to nuclear transfer. For the direct injection approach to nuclear transfer, the whole totipotent mammalian cell may be injected into the NT oocyte, or alternatively, a nucleus isolated from the totipotent mammalian cell may be injected into the NT oocyte. Such an isolated nucleus may be surrounded by nuclear membrane only, or the isolated nucleus may be surrounded by nuclear membrane and plasma membrane in any proportion. The NT oocyte may be pre-treated to enhance the strength of its plasma membrane, such as by incubating the NT oocyte in sucrose prior to injection of the nuclear donor.

For the purposes of the present invention, the term “embryo” or “embryonic” as used herein refers to a developing cell mass that has not implanted into the uterine membrane of a maternal host. Hence, the term “embryo” as used herein can refer to a fertilized oocyte, a cybrid (defined herein), a pre-blastocyst stage developing cell mass, a blastocyst stage embryo, a morula stage embryo, and/or any other developing cell mass that is at a stage of development prior to implantation into the uterine membrane of a maternal host. Embryos of the invention may not display a genital ridge. Hence, an “embryonic cell” is isolated from and/or has arisen from an embryo.

The term “fetus” as used herein refers to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species. The term “fetal cell” as used herein can refer to any cell isolated from and/or has arisen from a fetus or derived from a fetus. The term “non-fetal cell” is a cell that is not derived or isolated from a fetus.

The term “activation” refers to any materials and methods useful for stimulating a cell to divide before, during, and after a nuclear transfer step. An embryo obtained by a nuclear transfer procedure, that is, a combination of an NT oocyte and a nuclear donor cell or cell nucleus, may require stimulation in order to divide after a nuclear transfer has occurred. The invention pertains to any activation materials and methods known to a person of ordinary skill in the art. Although electrical pulses are sometimes sufficient for stimulating activation of nuclear transfer-derived embryos, other means are sometimes useful or necessary for proper activation. Chemical materials and methods useful for activating embryos are described below in other preferred embodiments of the invention.

Examples of non-electrical means for activation include agents such as ethanol; inositol trisphosphate (IP₃); Ca⁺⁺ ionophores (e.g., ionomycin) and protein kinase inhibitors (e.g., 6-dimethylaminopurine (DMAP)); temperature change; protein synthesis inhibitors (e.g., cyclohexamide); phorbol esters such as phorbol 12-myristate 13-acetate (PMA); mechanical techniques; and thapsigargin. The invention includes any activation techniques known in the art. See, e.g., U.S. Pat. No. 5,496,720 and U.S. Pat. No. 6,011,197, entitled “Parthenogenic Oocyte Activation,” incorporated by reference herein in their entirety, including all figures, tables, and drawings.

The term “totipotent” as used herein in reference to embryos refers to embryos that can develop into a live born animal.

The term “cloned” as used herein refers to a cell, embryonic cell, fetal cell, and/or animal cell having a nuclear DNA sequence that is substantially similar or identical to the nuclear DNA sequence of another cell, embryonic cell, fetal cell, and/or animal cell. The terms “substantially similar” and “identical” are described herein. The cloned embryo can arise from one nuclear transfer, or alternatively, the cloned embryo can arise from a cloning process that includes at least one re-cloning step.

The term “substantially similar” as used herein in reference to nuclear DNA sequences refers to two nuclear DNA sequences that are nearly identical. The two sequences may differ by copy error differences that normally occur during the replication of a nuclear DNA. Substantially similar DNA sequences are preferably greater than 97% identical, more preferably greater than 98% identical, and most preferably greater than 99% identical. The term “identity” is used herein in reference to nuclear DNA sequences can refer to the same usage of the term in reference to amino acid sequences, which is described previously herein.

The term “maturation” as used herein refers to process in which an oocyte is incubated in a medium in vitro. Oocytes can be incubated with multiple media well known to a person of ordinary skill in the art. See, e.g., Saito et al., 1992, Roux's Arch. Dev. Biol. 201: 134-141 for bovine organisms and Wells et al., 1997, Biol. Repr. 57: 385-393 for ovine organisms, both of which are incorporated herein by reference in their entireties including all figures, tables, and drawings. Maturation media can comprise multiple types of components, including microtubule and/or microfilament inhibitors (e.g., cytochalasin B). Other examples of components that can be incorporated into maturation media are discussed in WO 97/07668, entitled “Unactivated Oocytes as Cytoplast Recipients for Nuclear Transfer,” Campbell & Wilmut, published on Mar. 6, 1997, hereby incorporated herein by reference in its entirety, including all figures, tables, and drawings. The time of maturation can be determined from the time that an oocyte is placed in a maturation medium and the time that the oocyte is then utilized in a nuclear transfer procedure.

The term “cybrid” as used herein refers to a construction where an entire nuclear donor is translocated into the cytoplasm of a recipient oocyte. See, e.g., In Vitro Cell. Dev. Biol. 26: 97-101 (1990).

The term “canid” as used herein refers to any animal of the family Canidae. Preferably, a canid is a wolf, a jackal, a fox, and a domestic dog. The term “felid” as used herein refers to any animal of the family Felidae. Preferably, a felid is a lion, a tiger, a leopard, a cheetah, a cougar, and a domestic cat. The term “murid” as used herein refers to any animal of the family Muridae. Preferably, a murid is a mouse and a rat. The term “leporid” as used herein refers to any animal of the family Leporidae. Preferably, a leporid is a rabbit. The term “ursid” as used herein refers to any animal of the family Ursidae. Preferably, a ursid is a bear. The term “mustelid” as used herein refers to any animal of the family Mustelidae. Preferably, a mustelid is a weasel, a ferret, an otter, a mink, and a skunk. The term “primate” as used herein refers to any animal of the Primate order. Preferably, a primate is an ape, a monkey, a chimpanzee, and a lemur.

The term “ungulate” as used herein refers to any animal of the polyphyletic group formerly known as the taxon Ungulata. Preferably, an ungulate is a camel, a hippopotamus, a horse, a tapir, and an elephant. Most preferably, an ungulate is a sheep, a cow, a goat, and a pig. The term “ovid” as used herein refers to any animal of the family Ovidae. Preferably, an ovid is a sheep. The term “suid” as used herein refers to any animal of the family Suidae. Preferably, a suid is a pig or a boar. The term “equid” as used herein refers to any animal of the family Equidae. Preferably, an equid is a zebra or an ass. Most preferably, an equid is a horse. The term “caprid” as used herein refers to any animal of the family Caprinae. Preferably, a caprid is a goat. The term “cervid” as used herein refers to any animal of the family Cervidae. Preferably, a cervid is a deer.

The term “bovine” as used herein refers to a family of ruminants belonging to the genus Bos or any closely related genera of the family Bovidae. The family Bovidae includes true antelopes, oxen, sheep, and goats, for example. Preferred bovine animals are the cow and ox. Especially preferred bovine species are Bos taurus, Bos indicus. and Bos buffaloes. Other preferred bovine species are Bos primigenius and Bos longifrons.

The term “totipotent” as used herein in reference to cells refers to a cell that gives rise to all of the cells in a developing cell mass, such as an embryo, fetus, and animal. In preferred embodiments, the term “totipotent” also refers to a cell that gives rise to all of the cells in an animal. A totipotent cell can give rise to all of the cells of a developing cell mass when it is utilized in a procedure for creating an embryo from one or more nuclear transfer steps. An animal may be an animal that functions ex utero. An animal can exist, for example, as a live born animal. Totipotent cells may also be used to generate incomplete animals such as those useful for organ harvesting, e.g., having genetic modifications to eliminate growth of a head such as by manipulation of a homeotic gene.

The term “totipotent” as used herein is to be distinguished from the term “pluripotent.” The latter term refers to a cell that differentiates into a sub-population of cells within a developing cell mass, but is a cell that may not give rise to all of the cells in that developing cell mass. Thus, the term “pluripotent” can refer to a cell that cannot give rise to all of the cells in a live born animal.

The term “totipotent” as used herein is also to be distinguished from the term “chimer” or “chimera.” The latter term refers to a developing cell mass that comprises a sub-group of cells harboring nuclear DNA with a significantly different nucleotide base sequence than the nuclear DNA of other cells in that cell mass. The developing cell mass can, for example, exist as an embryo, fetus, and/or animal.

The term “confluence” as used herein refers to a group of cells where a large percentage of the cells are physically contacted with at least one other cell in that group. Confluence may also be defined as a group of cells that grow to a maximum cell density in the conditions provided. For example, if a group of cells can proliferate in a monolayer and they are placed in a culture vessel in a suitable growth medium, they are confluent when the monolayer has spread across a significant surface area of the culture vessel. The surface area covered by the cells preferably represents about 50% of the total surface area, more preferably represents about 70% of the total surface area, and most preferably represents about 90% of the total surface area. Nuclear donor cells can be obtained from confluent cultures.

In preferred embodiments, (1) the nuclear donor cell is selected from the group consisting of non-embryonic cell, a non-fetal cell, a differentiated cell, a somatic cell, an embryonic cell, a fetal cell, an embryonic stem cell, a primordial germ cell, a genital ridge cell, an amniotic cell, a fetal fibroblast cell, an ovarian follicular cell, a cumulus cell, an hepatic cell, an endocrine cell, an endothelial cell, an epidermal cell, an epithelial cell, a fibroblast cell, a hematopoletic cell, a keratinocyte, a renal cell, a lymphocyte, a melanocyte, a mussel cell, a myeloid cell, a neuronal cell, an osetoblast, a mesenchymal cell, a mesodermal cell, an adherent cell, a cell isolated from an asynchronous population of cells, and a cell isolated from a synchronized population of cells where the synchronous population is not arrested in the G₀ state of the cell cycle.

The term “primordial germ cell” as used herein refers to a diploid somatic cell capable of becoming a germ cell. Primordial germ cells can be isolated from the genital ridge of a developing cell mass. The genital ridge is a section of a developing cell mass that is well-known to a person of ordinary skill in the art. See, e.g., Strelchenko, 1996, Theriogenology 45: 130-141 and Lavoir 1994, J. Reprod. Dev. 37: 413-424.

The terms “embryonic germ cell” and “EG cell” as used herein refers to a cultured cell that has a distinct flattened morphology and can grow within monolayers in culture. An EG cell may be distinct from a fibroblast cell. This EG cell morphology is to be contrasted with cells that have a spherical morphology and form multicellular clumps on feeder layers. Embryonic germ cells may not require the presence of feeder layers or presence of growth factors in cell culture conditions. Embryonic germ cells may also grow with decreased doubling rates when these cells approach confluence on culture plates. Embryonic germ cells of the invention may be totipotent.

Embryonic germ cells may be established from a cell culture of nearly any type of precursor cell. Examples of precursor cells are discussed herein, and a preferred precursor cell for establishing an embryonic germ cell culture is a genital ridge cell from a fetus. Genital ridge cells are preferably isolated from procine fetuses where the fetus is between 20 days and parturition, between 30 days and 100 days, more preferably between 35 days and 70 days and between 40 days and 60 days, and most preferably about a 55 day fetus. An age of a fetus can be determined as described above. The term “about” with respect to fetuses can refer to plus or minus five days. As described herein, EG cells may be physically isolated from a primary culture of cells, and these isolated EG cells may be utilized to establish a cell culture that eventually forms a homogenous or nearly homogenous line of EG cells.

The term “embryonic stem cell” as used herein refers to pluripotent cells isolated from an embryo that are maintained in in vitro cell culture. Embryonic stem cells may be cultured with or without feeder cells. Embryonic stem cells can be established from embryonic cells isolated from embryos at any state of development, including blastocyst stage embryos and pre-blastocyst stage embryos. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335, both of which are incorporated herein by reference in their entireties, including all figures, tables, and drawings.

The term “ovarian follicular cell” as used herein refers to a cultured or non-cultured cell obtained from an ovarian follicle, other than an oocyte. Follicular cells may be isolated from ovarian follicles at any stage of development, including primordial follicles, primary follicles, secondary follicles, growing follicles, vesicular follicles, maturing follicles, mature follicles, and graafian follicles. Furthermore, follicular cells may be isolated when an oocyte in an ovarian follicle is immature (i.e., an oocyte that has not progressed to metaphase II) or when an oocyte in an ovarian follicle is mature (i.e., an oocyte that has progressed to metaphase II or a later stage of development). Preferred follicular cells include, but are not limited to, pregranulosa cells, granulosa cells, theca cells, columnar cells, stroma cells, theca interna cells, theca externa cells, mural granulosa cells, luteal cells, and corona radiata cells. Particularly preferred follicular cells are cumulus cells. Various types of follicular cells are known and can be readily distinguished by those skilled in the art. See, e.g., Laboratory Production of Cattle Embryos, 1994, Ian Gordon, CAB International; Anatomy and Physiology of Farm Animals (5th ed.), 1992, R. D. Frandson and T. L. Spurgeon, Lea & Febiger, each of which is incorporated herein by reference in its entirety including all figures, drawings, and tables. Individual types of follicular cells may be cultured separately, or a mixture of types may be cultured together.

The term “amniotic cell” as used herein refers to any cultured or non-cultured cell isolated from amniotic fluid. Examples of methods for isolating and culturing amniotic cells are discussed in Bellow et al., 1996, Theriogenology 45: 225; Garcia & Salaheddine, 1997, Theriogenology 47: 1003-1008; Liebo & Rail. 1990, Theriogenology 33: 531-552; and Vos et al., 1990, Vet. Rec. 127: 502-504, each of which is incorporated herein by reference in its entirety, including all figures tables and drawings. Particularly preferred are cultured amniotic cells that do not display a fibroblast-like morphology. The skilled artisan will understand that amniotic cells may be both maternal cells and fetal cells. Thus, preferred amniotic cells also include fetal fibroblast cells. The terms “fibroblast,” fibroblast-like,” “fetal,” and “fetal fibroblast” are defined hereafter.

The terms “fibroblast-like” and “fibroblast” as used herein refer to cultured cells that have a distinct flattened morphology and that are able to grow within monolayers in culture.

The term “fetal fibroblast cell” as used herein refers to any differentiated fetal cell having a fibroblast appearance. While fibroblasts characteristically have a flattened appearance when cultured on culture media plates, fetal fibroblast cells can also have a spindle-like morphology. Fetal fibroblasts may require density limitation for growth, may generate type I collagen, and may have a finite life span in culture of approximately fifty generations. Preferably, fetal fibroblast cells rigidly maintain a diploid chromosomal content. For a description of fibroblast cells, see, e.g. Culture of Animal Cells: a manual of basic techniques (3^{rd} edition), 1994, R. I. Freshney (ed), Wiley-Liss, Inc., incorporated herein by reference in its entirety, including all figures, tables, and drawings.

The terms “morphology” and “cell morphology” as used herein refer to form, structure, and physical characteristics of cells. For example, one cell morphology is significant levels of alkaline phosphatase, and this cell morphology can be identified by determining whether a cell stains appreciably for alkaline phosphatase. Another example of a cell morphology is whether a cell is flat or round in appearance when cultured on a surface or in the presence of a layer of feeder cells. Many other cell morphologies are known to a person of ordinary skill in the art and are cell morphologies are readily identifiable using materials and methods well known to those skilled in the art. See, e.g., Culture of Animal Cells: a manual of basic techniques (3^{rd} edition), 1994, R. I. Freshney (ed.). Wiley-Liss, Inc.

The term “cumulus cell” as used herein refers to any cultured or non-cultured cell isolated from cells and/or tissue surrounding an oocyte. Persons skilled in the art can readily identify cumulus cells. Examples of methods for isolating and/or culturing cumulus cells are discussed in Damiani et al., 1996, Mol. Reprod. Dev. 45: 521-534; Long et al., 1994, J. Reprod. Fert. 102: 361-369; and Wakayama et al., 1998, Nature 394: 369-373, each of which is incorporated herein by reference in its entireties, including all figures, tables, and drawings. Cumulus cells may be isolated from ovarian follicles at any stage of development, including primordial follicles, primary follicles, secondary follicles, growing follicles, vesicular follicles, maturing follicles, mature follicles, and graafian follicles. Cumulus cells may be isolated from oocytes in a number of manners well known to a person of ordinary skill in the art. For example, cumulus cells can be separated from oocytes by pipeting the cumulus cell/oocyte complex through a small bore pipette, by exposure to hyaluronidase, or by mechanically disrupting (e.g. vortexing) the cumulus cell/oocyte complex. Additionally, exposure to Ca⁺⁺/Mg⁺⁺ free media can remove cumulus from mature and/or immature oocytes. Also, cumulus cell cultures can be established by placing mature and/or immature oocytes in cell culture media Once cumulus cells are removed from media containing increased LH/FSH concentrations, they can to attach to the culture plate.

The term “hepatic cell” as used herein refers to any cultured or non-cultured cell isolated from a liver. Particularly preferred hepatic cells include, but are not limited to, an hepatic parenchymal cell, a Küpffer cell, an Ito cell, an hepatocyte, a fat-storing cell, a pit cell, and an hepatic endothelial cell. Persons skilled in the art can readily identify the various types of hepatic cells. See, e.g., Regulation of Hepatic Metabolism, 1986, Thurman et al. (eds.), Plenum Press, which is incorporated herein by reference in its entirety including all figures, drawings, and tables.

The term “asynchronous population” as used herein refers to cells that are not arrested at any one stage of the cell cycle. Many cells can progress through the cell cycle and do not arrest at any one stage, while some cells can become arrested at one stage of the cell cycle for a period of time. Some known stages of the cell cycle are G₀, G₁, S, G₂, and M. An asynchronous population of cells is not manipulated to synchronize into any one or predominantly into any one of these phases. Cells can be arrested in the G₀ stage of the cell cycle, for example, by utilizing multiple techniques known in the art, such as by serum deprivation. Examples of methods for arresting non-immortalized cells in one part of the cell cycle are discussed in WO 97/07669, entitled “Quiescent Cell Populations for Nuclear Transfer,” hereby incorporated herein by reference in its entirety, including all figures, tables, and drawings.

The terms “synchronous population” and “synchronizing” as used herein refer to a fraction of cells in a population that are arrested (i.e., the cells are not dividing) in a discreet stage of the cell cycle. Synchronizing a population of cells, by techniques such as serum deprivation, may render the cells quiescent. The term “quiescent” is defined below. Preferably, about 50% of the cells in a population of cells are arrested in one stage of the cell cycle, more preferably about 70% of the cells in a population of cells are arrested in one stage of the cell cycle, and most preferably about 90% of the cells in a population of cells are arrested in one stage of the cell cycle. Cell cycle stage can be distinguished by relative cell size as well as by a variety of cell markers well known to a person of ordinary skill in the art. For example, cells can be distinguished by such markers by using flow cytometry techniques well known to a person of ordinary skill in the art. Alternatively, cells can be distinguished by size utilizing techniques well known to a person of ordinary skill in the art, such as by the utilization of a light microscope and a micrometer, for example.

In yet another aspect, the present invention relates to cells and cell lines derived from the embryos and/or the reprogrammed cells described herein; and to uses thereof in cellular and tissue therapies.

### BRIEF DESCRIPTION OF THE DRAWINGS

FIG. 1 provides a schematic representation of remodeling of somatic chromatin by remodeling factors such as nucleoplasmin or polyglutamic acid.

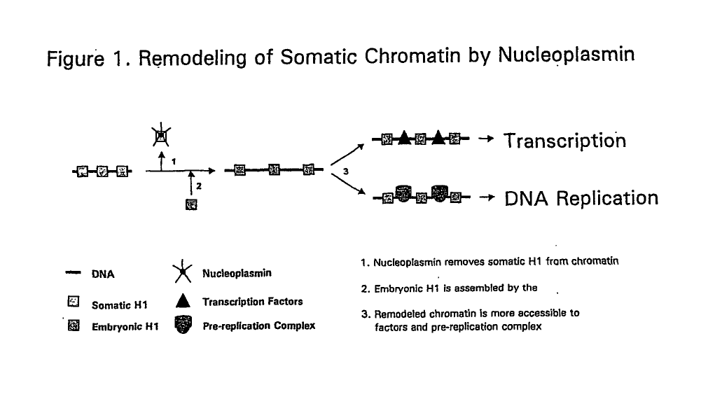

FIG. 2 provides a schematic representation of remodeling of somatic chromatin by cyclin A-dependent kinase.

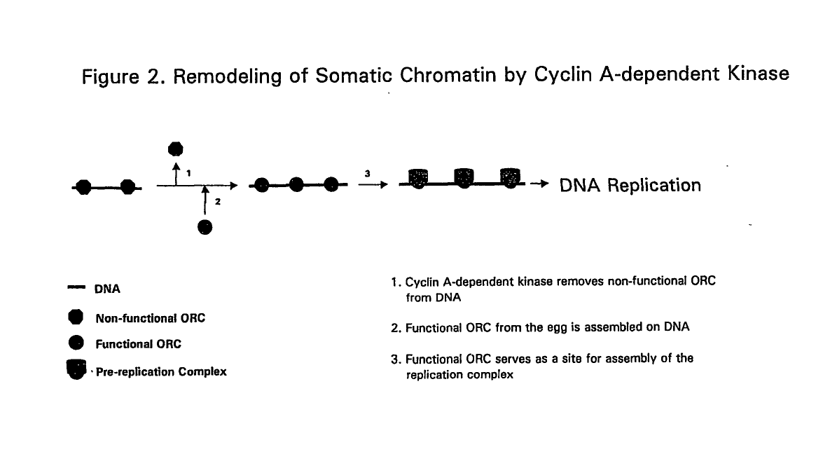

FIG. 3 provides a schematic representation of microinjection of nucleoplasmin before or after nuclear transfer and remodeling of somatic nuclei before nuclear transfer.

FIG. 4 provides a schematic representation of remodeling of somatic nuclei with extracts from Xenopus oocytes and eggs before nuclear transfer.

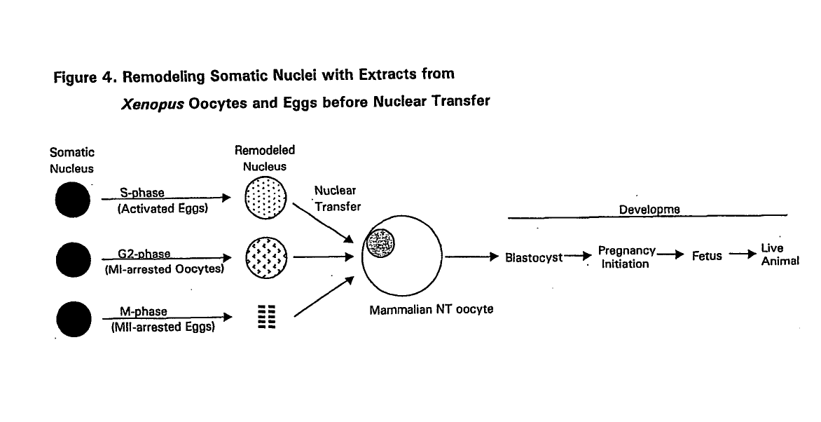

### DETAILED DESCRIPTION OF THE PREFERRED EMBODIMENTS

An important event in cloning procedures is the introduction of the donor nucleus into the recipient NT oocyte, a process known as nuclear transfer. Changes in both nuclear and chromatin structure occur following transfer of the pre-S-phase nucleus into the NT oocyte cytoplasm, including nuclear envelope breakdown and chromosome condensation. See, e.g., Bordignon et al., Dev. Biol. 233: 192-203 (2001). These changes occur because the NT oocyte is derived by enucleation of an oocyte in metaphase of meiosis II. Active Cdc2-cyclin B, also known as maturation promoting factor or MPF, may facilitate many of the changes in nuclear and chromatin structure that are associated with metaphase arrest and thus may induce these changes in donor nuclei following nuclear transfer. From the perspective of the donor nucleus, however, these events are premature given that each would occur only at the next mitotic metaphase.

Thus, bypassing S- and G2-phases of the cell cycle may limit the donor nucleus' ability to undergo remodeling by the bovine NT oocyte. On the other hand, the transition from an interphase nucleus to metaphase chromosomes may contribute to reprogramming of the bovine somatic DNA. Thus, the present inventors realized that two separate problems may exist. First, pre-S-phase nuclei may not be adequately prepared to enter a metaphase environment; and second, once in that environment, the duration of exposure to reprogramming activities may be insufficient for conversion to the totipotent state. But these problems may not be mutually exclusive and aspects of each may contribute to cloning inefficiencies.

In short, then, the complete reprogramming of the somatic nucleus facilitates normal development of the cloned embryo, but factors that facilitate remodeling are limiting or absent from the bovine egg. Thus, production of a cloned animal is a relatively rare event. By supplementing the NT oocyte chromatin with additional remodeling factors, one may facilitate the required reprogramming, and dramatically increase the efficiencies seen in nuclear transfer procedures.

Nuclear Donors for Nuclear Transfer

For nuclear transfer techniques, a donor cell may be separated from a growing cell mass, isolated from a primary cell culture, or isolated from a cell line. The entire cell may be placed in the perivitelline space of a recipient oocyte or may be directly injected into the recipient oocyte by aspirating the nuclear donor into a needle, placing the needle into the recipient oocyte, releasing the nuclear donor and removing the needle without significantly disrupting the plasma membrane of the oocyte. Also, a nucleus (e.g., karyoplast) may be isolated from a nuclear donor and placed into the perivitelline space of a recipient oocyte or may be injected directly into a recipient oocyte, for example.

Recipient NT Oocytes

A recipient NT oocyte is typically an oocyte with a portion of its ooplasm removed, where the removed ooplasm comprises the oocyte nucleus. Enucleation techniques are well known to a person of ordinary skill in the art. See e.g., Nagashima et al, 1997, Mol. Reprod. Dev. 48: 339-343; Nagashima et al, 1992, J. Reprod. Dev. 38: 37-78; Prather et al., 1989, Biol. Reprod. 41: 414-418; Prather et al., 1990, J. Exp. Zool. 255: 355-358; Saito et al, 1992, Assis. Reprod. Tech. Andro. 259: 257-266; and Terlouw et al., 1992, Theriogenology 37: 309, each of which is incorporated herein by reference in its entirety including all figures, tables, and drawings.

NT oocytes can be isolated from either oviducts and/or ovaries of live animals by oviductal recovery procedures or transvaginal oocyte recovery procedures well known in the art and described herein. Furthermore, oocytes can be isolated from deceased animals. For example, ovaries can be obtained from abattoirs and oocytes can be aspirated from these ovaries. The oocytes can also be isolated from the ovaries of a recently sacrificed animal or when the ovary has been frozen and/or thawed.

NT oocytes can be matured in a variety of media well known to a person of ordinary skill in the art. One example of such a medium suitable for maturing oocytes is depicted in an exemplary embodiment described hereafter. Oocytes can be successfully matured in this type of medium within an environment comprising 5% CO₂ at 39° C. Oocytes may be cryopreserved and then thawed before placing the oocytes in maturation medium. Cryopreservation procedures for cells and embryos are well known in the art as discussed herein.

Components of an oocyte maturation medium can include molecules that arrest oocyte maturation. Examples of such components are 6-dimethylaminopurine (DMAP) and isobutylmethylxanthine (IBMX. IBMX has been reported to reversibly arrest oocytes, but the efficiencies of arrest maintenance are quite low. See, e.g., Rose-Hellkant and Bavister, 1996, Mol. Reprod. Develop. 44: 241-249. However, oocytes may be arrested at the germinal vesicle stage with a relatively high efficiency by incubating oocytes at 31° C. in an effective concentration of IBMX. Preferably, oocytes are incubated the entire time that oocytes are collected. Concentrations of IBMX suitable for arresting oocyte maturation are 0.01 mM to 20 mM IBMX, preferably 0.05 mM to 10 mM IBMX, and more preferably about 0.1 mM IBMX to about 0.5 mM IBMX, and most preferably 0.1 mM IBMX to 0.5 mM IBMX. In certain embodiments, oocytes can be matured in a culture environment having a low oxygen concentration, such as 5% O₂, 5-10% CO₂, and 85-90% N₂.

A nuclear donor cell and a recipient NT oocyte can arise from the same species or different species. For example, a totipotent porcine cell can be inserted into a porcine enucleated oocyte. Alternatively, a totipotent wild boar cell can be inserted into a domesticated porcine oocyte. Any nuclear donor/recipient oocyte combinations are envisioned by the invention. Preferably the nuclear donor and recipient oocyte from the same specie. Cross-species nuclear transfer techniques can be utilized to produce cloned animals that are endangered or extinct.

NT oocytes can be activated by electrical and/or non-electrical means before, during, and/or after a nuclear donor is introduced to recipient oocyte. For example, an oocyte can be placed in a medium containing one or more components suitable for non-electrical activation prior to fusion with a nuclear donor. Also, a cybrid can be placed in a medium containing one or more components suitable for non-electrical activation. Activation processes are discussed in greater detail hereafter.

Injection/Fusion of Nuclear Donors into NT Oocytes

A nuclear donor can be translocated into an NT oocyte using a variety of materials and methods that are well known to a person of ordinary skill in the art. In one example, a nuclear donor may be directly injected into a recipient NT oocyte. This direct injection can be accomplished by gently pulling a nuclear donor into a needle, piercing a recipient NT oocyte with that needle, releasing the nuclear donor into the NT oocyte, and removing the needle from the NT oocyte without significantly disrupting its membrane. Appropriate needles can be fashioned from glass capillary tubes, as defined in the art and specifically by publications incorporated herein by reference.

In another example, at least a portion of plasma membrane from a nuclear donor and recipient NT oocyte can be fused together by utilizing techniques well known to a person of ordinary skill in the art. See, Willadsen, 1986, Nature 320: 63-65, hereby incorporated herein by reference in its entirety including all figures, tables, and drawings. Typically, lipid membranes can be fused together by electrical and chemical means, as defined previously and in other publications incorporated herein by reference.

Examples of non-electrical means of cell fusion involve incubating the cells to be fused in solutions comprising polyethylene glycol (PEG), and/or Sendai virus. PEG molecules of a wide range of molecular weight can be utilized for cell fusion.

Processes for fusion that are not explicitly discussed herein can be determined without undue experimentation. For example, modifications to cell fusion techniques can be monitored for their efficiency by viewing the degree of cell fusion under a microscope. The resulting embryo can then be cloned and identified as a totipotent embryo by the same methods as those previously described herein for identifying totipotent cells, which can include tests for selectable markers and/or tests for developing an animal.

Activation of Nuclear Transfer-Derived Embryos

Methods of activating NT oocytes and cybrids are known to those of ordinary skill in the art. See, U.S. Pat. No. 5,496,720, “Parthenogenic Oocyte Activation,” Susko-Parrish et al., issued on Mar. 5, 1996, hereby incorporated by reference herein in its entirety including all figures, tables, and drawings.

Both electrical and non-electrical processes can be used for activating cells (e.g., oocytes and cybrids). Although use of a non-electrical means for activation is not always necessary, non-electrical activation can enhance the developmental potential of cybrids, particularly when young oocytes are utilized as recipients.

Examples of electrical techniques for activating cells are well known in the art. See, WO 98/16630, published on Apr. 23, 1998, Piedrahita and Blazer, hereby incorporated herein in its entirety including all figures, tables, and drawings, and U.S. Pat. Nos. 4,994,384 and 5,057,420. Non-electrical means for activating cells can include any method known in the art that increases the probability of cell division. Examples of non-electrical means for activating a nuclear donor and/or recipient can be accomplished by introducing cells to ethanol; inositol trisphosphate (IP₃); Ca²⁺ ionophore and protein kinase inhibitors such as 6-dimethylaminopurine; temperature change; protein synthesis inhibitors (e.g., cycloheximide); phorbol esters such as phorbol 12-myristate 13-acetate (PMA); mechanical techniques, thapsigargin, and sperm factors. Sperm factors can include any component of a sperm that enhance the probability for cell division. Other non-electrical methods for activation include subjecting the cell or cells to cold shock and/or mechanical stress.

Examples of preferred protein kinase inhibitors are protein kinase A, G, and C inhibitors such as 6-dimethylaminopurine (DMAP), staurosporin, 2-aminopurine, sphingosine. Tyrosine kinase inhibitors may also be utilized to activate cells.

Activation materials and methods that are not explicitly discussed herein can be identified by modifying the specified conditions defined in the exemplary protocols described hereafter and in U.S. Pat. No. 5,496,720.

Manipulation of Embryos Resulting from Nuclear Transfer

An embryo resulting from a nuclear transfer process can be manipulated in a variety of manners. The invention relates to cloned embryos that arise from at least one nuclear transfer. Exemplary embodiments of the invention demonstrate that two or more nuclear transfer procedures may enhance the efficiency for the production of totipotent embryos. Exemplary embodiments indicate that incorporating two or more nuclear transfer procedures into methods for producing cloned totipotent embryos may enhance placental development. In addition, increasing the number of nuclear transfer cycles involved in a process for producing totipotent embryos may represent a necessary factor for converting non-totipotent cells into totipotent cells. An effect of incorporating two or more nuclear transfer cycles upon totipotency of resulting embryos is a surprising result, which was not previously identified or explored in the art.

Incorporating two or more nuclear transfer cycles into methods for cloned totipotent embryos can provide further advantages. Incorporating multiple nuclear transfer procedures into methods for establishing cloned totipotent embryos provides a method for multiplying the number of cloned totipotent embryos.

When multiple nuclear transfer procedures are utilized for the formation of a cloned totipotent embryo, NT oocytes that have been matured for any period of time can be utilized as recipients in the first, second or subsequent nuclear transfer procedures. For example, if a first nuclear transfer and then a second nuclear transfer are performed, the first nuclear transfer can utilize an NT oocyte that has been matured for about 24 hours as a recipient and the second nuclear transfer may utilize an NT oocyte that has been matured for less than about 36 hours as a recipient. Alternatively, the first nuclear transfer may utilize an NT oocyte that has been matured for about 36 hours as a recipient and the second nuclear transfer may utilize an NT oocyte that has been matured for greater than about 24 hours as a recipient for a two-cycle nuclear transfer regime. In addition, both nuclear transfer cycles may utilize NT oocytes that have been matured for about the same number of hours as recipients in a two-cycle nuclear transfer regime.

For nuclear transfer techniques that incorporate two or more nuclear transfer cycles, one or more of the nuclear transfer cycles may be preceded, followed, and/or carried out simultaneously with an activation step. As defined previously herein, an activation step may be accomplished by electrical and/or non-electrical means as defined herein. Exemplified embodiments described hereafter describe nuclear transfer techniques that incorporate an activation step after one nuclear transfer cycle. However, an activation step may also be carried out at the same time as a nuclear transfer cycle (e.g., simultaneously with the nuclear transfer cycle) and/or an activation step may be carried out prior to a nuclear transfer cycle. Cloned totipotent embryos resulting from a nuclear transfer cycle can be (1) disaggregated or (2) allowed to develop further.

If embryos are disaggregated, disaggregated embryonic derived cells can be utilized to establish cultured cells. Any type of embryonic cell can be utilized to establish cultured cells. These cultured cells are sometimes referred to as embryonic stem cells or embryonic stem-like cells in the scientific literature. The embryonic stem cells can be derived from early embryos, morulae, and blastocyst stage embryos. Multiple methods are known to a person of ordinary skill in the art for producing cultured embryonic cells. These methods are enumerated in specific references previously incorporated by reference herein.

Embryonic stem cells and/or other cell lines prepared from the methods described herein may be used for a variety of purposes well known to those of skill in the art. These uses include, but are not limited to: generating transgenic non-human animals for models of specific human genetic diseases; and generation of non-human or human tissue or models for any human genetic disease for which the responsible gene has been cloned; generation of non-human or human cells or tissue for cellular or tissue transplantation. By manipulating culture conditions, embryonic stem cells, human and non-human, can be induced to differentiate to specific cell types, such as blood cells, neuron cells, or muscle cells. Alternatively, embryonic stem cells, human and non-human can be allowed to differentiate in tumors in SCID mice, the tumors can be disassociated, and the specific differentiated cell types of interest can be selected by the usage of lineage specific markers through the use of fluorescent activated cell sorting (FACS) or other sorting method or by direct microdissection of tissues of interest. These differentiated cells could then be transplanted back to an adult animal to treat specific diseases, such as hematopoietic disorders, endocrine deficiencies, degenerative neurological disorders or hair loss.

If embryos are allowed to develop into a fetus in utero, cells isolated from that developing fetus can be utilized to establish cultured cells. In preferred embodiments, primordial germ cells, genital ridge cells, and fetal fibroblast cells can be isolated from such a fetus. Cultured cells having a particular morphology that is described herein can be referred to as embryonic germ cells (EG cells). These cultured cells can be established by utilizing culture methods well known to a person of ordinary skill in the art. Such methods are enumerated in publications previously incorporated herein by reference and are discussed herein. In particularly preferred embodiments, Streptomyces griseus protease can be used to remove unwanted cells from the embryonic germ cell culture.

Cloned totipotent embryos resulting from nuclear transfer can also be manipulated by cryopreserving and/or thawing the embryos. See, e.g., Nagashima et al., 1989, Japanese J. Anim. Reprod. 35: 130-134 and Feng et al., 1991, Theriogenology 35: 199, each of which is incorporated herein by reference in its entirety including all tables, figures, and drawings. Other embryo manipulation methods include in vitro culture processes; performing embryo transfer into a maternal recipient; disaggregating blastomeres for nuclear transfer processes; disaggregating blastomeres or inner cell mass cells for establishing cell lines for use in nuclear transfer procedures; embryo splitting procedures; embryo aggregating procedures; embryo sexing procedures; and embryo biopsying procedures. The exemplary manipulation procedures are not meant to be limiting and the invention relates to any embryo manipulation procedure known to a person of ordinary skill in the art.

Culture of Nuclear Transfer-Embryos In vitro

Cloning procedures discussed herein provide an advantage of culturing cells and embryos in vitro prior to implantation into a recipient female. Methods for culturing embryos in vitro are well known to those skilled in the art. See, e.g., Nagashima et al., 1997, Mol. Reprod. Dev. 48: 339-343; Petters & Wells, 1993, J. Reprod. Fert. (Suppl) 48: 61-73; Reed et al., 1992, Theriogenology 37: 95-109; and Dobrinsky et al., 1996, Biol. Reprod. 55: 1069-1074, each of which is incorporated herein by reference in its entirety, including all figures, tables, and drawings. In addition, exemplary embodiments for media suitable for culturing cloned embryos in vitro are described hereafter. Feeder cell layers may or may not be utilized for culturing cloned embryos in vitro. Feeder cells are described previously and in exemplary embodiments hereafter.

Development of Nuclear Transfer-Embryos In Utero

Cloned embryos can be cultured in an artificial or natural uterine environment after nuclear transfer procedures and embryo in vitro culture processes. Examples of artificial development environments are being developed and some are known to those skilled in the art. Components of the artificial environment can be modified, for example, by altering the amount of a component or components and by monitoring the growth rate of an embryo.

Methods for implanting embryos into the uterus of an animal are also well known in the art, as discussed previously. Preferably, the developmental stage of the embryo(s) is correlated with the estrus cycle of the animal.

Embryos from one specie can be placed into the uterine environment of an animal from another specie. For example it has been shown in the art that bovine embryos can develop in the oviducts of sheep. Stice & Keefer, 1993, “Multiple generational bovine embryo cloning,” Biology of Reproduction 48: 715-719. The invention relates to any combination of a embryo in any other ungulate uterine environment. A cross-species in utero development regime can allow for efficient production of cloned animals of an endangered species. For example, a wild boar embryo can develop in the uterus of a domestic porcine sow.

Once an embryo is placed into the uterus of a recipient female, the embryo can develop to term. Alternatively, an embryo can be allowed to develop in the uterus and then can be removed at a chosen time. Surgical methods are well known in the art for removing fetuses from uteri before they are born.

Use of Reprogramming Factors in Nuclear Transfer

As discussed above, there are numerous remodeling factors known in the art, including, but not limited to, ATP-dependent remodeling factors (e.g., SWI/SNF, ISWI, and ISWI homologs from yeast and Xenopus); non-ATP-dependent remodeling factors (e.g., nucleoplasmin and polyanionic molecules such as polyglutamic acid). Female germ cell extracts may provide the widest array of remodeling possibilities due to the repertoire of remodeling factors that they contain. If many remodeling factors are required for successful reprogramming, then female germ cell extracts provide an excellent environment for the coordination of these events. But if reprogramming requires the activity of a smaller number of remodeling factors, then the use of these factors alone, or in combination may be preferred in order to avoid potential toxicity from contaminating proteins. But either approach may facilitate the remodeling of donor nuclei.

Amphibian cells may be a particularly rich source of these supplemental reprogramming factors. For example, in Xenopus, the female germ cell, or oocyte, is normally arrested in G2-phase/prophase of meiosis I within the ovary of the adult frog. During this stage of meiotic arrest, oocyte growth or oogenesis occurs. Typically, it takes 3 months or more for a stage I Xenopus oocyte to become a fully-grown stage VI form. During this period, oocytes accumulate a stockpile of macromolecules and organelles that are required to support the rapid cell cycles in the early embryo. Fully-grown oocytes are then induced to complete meiosis I and enter a second stage of arrest in metaphase of meiosis II. This process of oocyte maturation occurs in response to secretion of progesterone from the surrounding follicle cells. The mature oocyte then passes down the oviduct and is released by the frog as an unfertilized egg. Upon fertilization, the egg is released from metaphase arrest and enters interphase, with the first mitotic cell cycle lasting approximately 90 minutes and the next 11, only 30 minutes each.

These early embryonic cell cycles consist of alternating S- and M-phases without G1- or G2-phases or gene transcription. The stockpile of components present within the oocyte and later within the egg not only supports these remarkably rapid embryonic cell cycles in vivo, but it also supports the simultaneous remodeling of thousands of somatic nuclei in vitro. Furthermore, cytoplasmic extracts from female germ cells isolated at different points within this developmental pathway may offer unique opportunities for reprogramming the somatic nucleus prior to nuclear transfer.

The present invention provides strategies for supplementing the remodeling capacity of the mammalian NT oocyte to improve development of the cloned embryo and improve the rates of successful cloning. These strategies are illustrated in the following sections.

Nucleoplasmin as a Remodeling Factor

The remodeling protein nucleoplasmin (NPL), can be injected into a mammalian NT oocyte before, during, or after nuclear transfer of a somatic cell into the NT oocyte. It is believed that nucleoplasmin facilitates the coordinate exchange of somatic proteins with egg proteins. This coordination of specific remodeling events, e.g., the exchange of somatic H1 for embryonic H1oo, may also facilitate the formation of higher-order chromatin structure in the donor nucleus.

In preferred embodiments, nucleoplasmin is prepared and somatic cells are grown as in Example 1, below. Donor nuclei can be incubated with NPL for various times over a concentration range that represents a 5-fold lower to a 5-fold higher concentration than that found in the Xenopus egg, and the time-dependent loss of histone H1 from chromatin over the range of NPL concentrations can be monitored. H1 levels may be determined by resolving acid extracted chromatin proteins by SDS-PAGE (Lu et al., J. Cell Sci., 110(Pt 21): 2745-58, 1997; Lu et al., Mol. Biol. Cell, 9(5): 163-76, 1998; Lu et al., Mol. Biol. Cell, 10(12): 4091-106, 1999). By such methods, conditions can rapidly be identified that result in the rapid and complete removal of H1 from donor nuclei. NPL-remodeled nuclei, and buffer-incubated control nuclei, may then be used for nuclear transfer.

Cyclin A-Dependent Kinases as Remodeling Factors

Cdc2/Cdk2-cyclin A (150 nM) can be used alone, or combined with other remodeling factors (e.g., the optimal concentration of NPL as determined by the methods described herein) to remodel somatic donor chromatin. It is believed that cyclin A-dependent kinases act to remove preexisting, non-functional origin recognition complex (“ORC”) proteins from chromatin, a necessary step in the remodeling process.

When both cyclin A-dependent kinases and NPL are used, the loss of both H1 and ORC proteins from chromatin can be monitored in time-course studies as described above. The time required for complete removal of these proteins is determined and used to incubate nuclei before nuclear transfer.

Cell Extracts as Remodeling Factors

Donor cells can be treated with the bacterial toxin streptolysin-O (SLO) or digitonin to permeabilize the plasma membrane but not the nuclear membrane. Without wanting to be bound by any particular theory, it is believed that this differential permeability of plasma and nuclear membranes accomplishes three goals—it may prevent the loss of important components from the nucleus during cell isolation; it may promote the release of diffusible cytoplasmic factors from the cell that may impede the reprogramming of somatic nuclei within the egg; and it may allow for the introduction of reprogramming factors from oocyte or egg extracts into the donor cell. It is believed that once within the permeable cells, these factors will be concentrated within the nucleus by an intact, functional nuclear envelope, and that reaching a threshold nuclear concentration may trigger key reprogramming events. Reprogramming factors may include known chromatin-remodeling proteins such as nucleoplasmin, protein kinases such as the cyclin-dependent kinases, or presently unknown factors that may be abundant in amphibian oocyte and egg extracts but not in mammalian eggs. Three different extracts can be used to remodel donor cell nuclei. Each is obtained from cells, preferably amphibian cells, and most preferably Xenopus cells, arrested at a different point within the mitotic or meiotic cell cycle, and therefore, each should modify nuclear and chromatin structure in unique and potentially important ways.

### EXAMPLE 1

### Bovine Nuclear Transfer

Oocytes aspirated from ovaries were matured overnight in maturation medium (Medium 199 (Biowhittaker, Inc.) supplemented with luteinizing hormone (10 IU/ml, Sigma), 1 mg/ml estradiol (Sigma) and 10% FBS) at 38.5° C. in a humidified CO₂ incubator. Typically, after 16-17 hours of maturation, the cumulus cell layer had expanded and the first polar bodies were extruded in approximately 70% of the oocytes. The oocytes were stripped of cumulus cells by vortexing in 0.5 ml of TL-HEPES. The chromatin was stained with Hoechst 33342 (5 μg/ml, Sigma) in TL-HEPES solution for 15 min. Oocytes were then enucleated in TL-HEPES solution under mineral oil.

A single nuclear donor cell was then inserted into the perivitelline space of the injected oocyte. Fusion of the cell and oocyte membranes was induced by electrofusion in a 500 μm chamber by applying an electrical pulse of 90V for 15 μs in an isotonic sorbitol solution (0.25 M) containing calcium acetate (0.1 mM), magnesium acetate (0.5 mM), and fatty acid free bovine serum albumin (BSA) (1 mg/ml, Sigma #A7030)(H 7.2) at 30° C. After 0-3 hr of culture in CR1aa (CR2) medium [Rosenkrans Cf Jr, 1994 #189] containing 3 mg/ml BSA, injection of NPL in buffer, polyglutamic acid (PGA) in buffer and/or buffer alone occurred using a PiezoDrill™ (Burleigh Instruments, Fishers, N.Y.). A glass injection tip (˜8-10 μm outside diameter) attached to the PiezoDrill was used to aspirate buffer solutions and expel into oocytes so as not to lyse the oocyte. Injection buffer consisted of 70 mM potassium chloride and 20 mM HEPES, pH 7.0. A volume of approximately 1/3 to ¼ the volume of the oocyte was injected. Oocyte injection occurred in calcium-free TL-HEPES. Following injection and approximately 4 hr post fusion, activation of the nuclear transfer embryos was induced by a 4 min exposure to 5 μM ionomycin (Ca²⁺-salt) (Sigma) in TL-HEPES, containing 1 mg/ml BSA, followed by a wash in TL-HEPES containing no ionomycin. The embryos were then incubated in CR2 medium containing 1.9 mM 6-dimethylaminopurine (DMAP, Sigma) for 4 hrs followed by a wash in TL-HEPES and then cultured in CR2 media with BSA (3 mg/ml) at 38.5° C. in a humidified 5% CO₂ incubator. Three days later the embryos were transferred to CR2 medium containing 10% FBS and cultured for 1-4 days.

### EXAMPLE 2

### Use of Nucleoplasmin

Nucleoplasmin (NPL) was purified from Xenopus eggs by using the following two methods:
1. Method described by Dingwall et al., Cell 30: 449-58 (1982) with modifications. EHSS was prepared and diluted in 2 volumes of buffer A (60 mM KCl, 15 mM NaCl, 1 mM β-mercaptoethanol, 0.5 mM spermidine, 0.15 mM spermine, 15 mM Tris-HCl, pH 7.4), heated at 80° C. for 10 min in a water bath, and centrifuged in a bench top centrifuge at 10,000 rpm for 5 min. The supernatant was pooled (˜40 ml total volume) and loaded onto a 14 cm by 1.6 cm (˜24 ml) Whatman DE52 DEAE-cellulose column (Whatman Inc. Clifton, N.J.) that had been equilibrated with buffer EQ (50 mM NaCl, 1 mM EDTA, 1 mM β-mercaptoethanol, 0.1 mM PMSF, 25 mM Tris-HCl, pH 7.5). The column was washed extensively with buffer EQ until the absorbance at 280 nm was back to baseline and then eluted with a linear NaCl gradient (42 ml+42 ml) increasing to 0.4 M in buffer EQ. Fractions containing nucleoplasmin were identified by SDS-PAGE and pooled, brought to 55% saturation with (NH₄)₂SO₄, and incubated overnight at 4° C. The mixture was centrifuged at 10,000 rpm for 30 min at 4° C., and the supernatant was taken and loaded onto a 17 cm by 1.0 cm (˜9 ml) phenyl sepharose 4LB column (Amersham Pharmacia Biotech, Piscataway, N.J.) equilibrated with buffer EL [1.5 M (NH₄)₂SO₄, 20 mM Tris-HCl, pH 7.6]. The column was washed extensively with buffer EL and then eluted with a linear gradient (22.5 ml+22.5 ml) of decreasing (NH₄)₂SO₄ to 0 M. The nucleoplasmin-containing fractions identified by SDS-PAGE were pooled, dialyzed against 20 mM NH₄HCO₃, and centrifuged to remove particulate material. The resultant supernatant was lyophilized and stored dry at −80° C. The identity of nucleoplasmin as the prominent protein in the lyophilized sample was confirmed by Western blotting with an anti-nucleoplasmin monoclonal antibody derived from the hybridoma clone, PA3C5 [Dilworth et al., Cell 51: 1009-18 (1987)]
2. Method described by Philpott et al., Cell 65: 569-78 (1991) with modifications. Mouse anti-nucleoplasmin monoclonal antibody was derived from the hybridoma clone PA3C5. The production and purification of the antibody was performed according to standard methods. (NH₄)₂SO₄ was added to the hybridoma culture supernatant to 55% saturation and kept at 4° C. overnight. The mixture was centrifuged at 3000 g for 30 min. The pellet was dissolved in D-PBS and filtered through a 0.45 μm filter. The solution was then applied to a 7 ml protein A sepharose CL-4B (Amersham Pharmacia Biotech) column. The column was washed with 20 column volumes of D-PBS and then the antibody was eluted with 0.1 M glycine buffer (pH 3.0) into 1/10 volume 1M Tris-HCl (pH 8.0). Peak fractions containing the antibody were concentrated with Amicon 10 centriprep protein concentrator. Concentrated protein was dialyzed against D-PBS overnight and then stored at 4° C. The purified nucleoplasmin antibody was conjugated to activated CNBr sepharose 4B resin (Amersham Pharmacia Biotech) using manufacturer's protocol. 2-3 ml of HSS extract was diluted with NET(+) buffer (150 mM NaCl, 5 mM EDTA, 1 μg/ml each of aprotinin, leupeptin, pepstatin A and chymostatin, 10 mM Na₄P₂O₇, 50 mM Tris-HCl, pH 7.5) to a final volume of 10 ml and loaded onto a 3.5 ml antibody coupled sepharose column. The column was then washed extensively (>5 bed volume) with NET(+) buffer and then eluted with 100 mM sodium citrate buffer (pH 3.0) containing 1 μg/ml each of aprotinin, leupeptin, pepstatin and chymostatin. The fractions were collected in the presence of 1/10 volume of 1 M Tris-HCl (pH 8.0). Fractions containing the elution peak were determined by spectrophotometer (280 nm) and pooled, concentrated with Amicon 10 Centriprep filer device to a final volume of ˜0.5 ml. The concentrated protein solution was transferred to a Slide-A-Lyzer dialysis cassette (Pierce) and dialyzed against 0.5×EB (extraction buffer, 1×=50 mM Hepes-KOH, pH 7.6, 50 mM KCl, S mM MgCl₂, 2 mM 2-mercaptoethanol) overnight. The resultant solution was retrieved and concentrated with a speed vacuum and stored at 4° C.

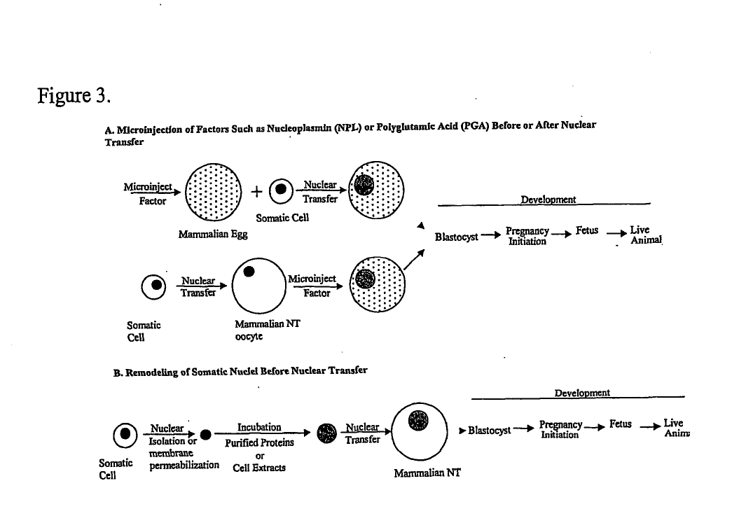

The purity of isolated nucleoplasmin was assessed by staining of the SDS-PAGE gels with Coomassie blue (FIG. 3). Dried samples were dissolved in EB before use. DC protein assay kit (Bio-Rad) or molar extinction coefficient of 13,980 M⁻¹cm ⁻¹ at 280 nm was used to determine the concentration of nucleoplasmin.

Adult and fetal bovine somatic cells for use as nuclear donors were grown to confluence. For post-nuclear transfer microinjection of remodeling factors, somatic cells were first fused with in vitro matured bovine NT oocytes and then injected with NPL. Alternatively, for pre-nuclear transfer microinjection of remodeling factors, bovine eggs are injected with NPL and then fused with somatic cells. The final concentration of NPL in the oocyte was approximately 500 ng/μl. A total volume of approximately 0.4 nl was injected into each egg.

Development of the cloned embryo was compared among two groups:
Group I: Control Nuclear Transfers—nuclear transfer of donor cells without microinjection of the oocyte; and
Group II: Nucleoplasmin (NPL)—injection of NPL into the oocyte after nuclear transfer.

The oocytes were then activated and the resultant embryos cultured in vitro. The percent of embryos developing to blastocyst were determined and compared among the four groups. Results for these groups are presented in Table 1:

TABLE 1
Blastocyst Development in Nucleoplasmin (NPL)-Injected
Eggs After Nuclear Transfer

| | | | Percent of Nuclear |
|---|---|---|---|
| | Number of | Number of | Transfers that |
| | Nuclear | Blastocysts | Develop to |
| | Transfers | Produced | Blastocyst |
| Control (no | 135 | 26 | 19.3* |
| injection) Nuclear | | | |
| Transfers | | | |
| NPL-injection of | 41 | 24 | 58.5* |
| Eggs After Nuclear | | | |
| Transfer | | | |

*Data from three separate experiments with two cell lines

These results show that, as previously described, mammalian oocytes show a limited ability to reprogram somatic cell nuclei in the absence of remodeling factors, as demonstrated by the low percentage of nuclear transfer embryos proceeding to blastocyst stage. This percentage can be dramatically increased by the addition of NPL into the mammalian oocyte.

After reaching the blastocyst stage, eleven of the group 2 blastocysts were transferred into the uterus of a bovine host, to determine if such embryos can support pregnancy development. Pregnant animals were checked at regular intervals post transfer for maintenance of the pregnancy. Of these eleven, three confirmed pregnancies (27%) were obtained, and eight failed to establish pregnancies.

While this example utilizes bovines by way of example, the person of ordinary skill in the art will realize that mammalian embryos of any mammalian species may be prepared similarly, including, but not limited to, ovines and porcines. And while this example utilized bovine fetal cells by way of example, other cells may also be used including, but not limited to, an embryonic cell, an adult cell, a somatic cell, a primordial cell, a fibroblast cell, a cumulus cell, an amniotic cell, or any transgenic cell, as described herein.

### EXAMPLE 3

### Use of Cyclin A-Dependent Kinase

The fusion proteins, GST-Xenopus cyclin A1 and GST-human Cdk2, are expressed and purified as described (Jackson et al., 1995). Xenopus GST-Cdc2 is expressed in E. coli and purified as described (Poon et al., 1993). Purified recombinant Cdc2 or Cdk2 (150 nM) and cyclin A (150 nM) in kinase buffer containing ATP phosphorylates purified histone H1 and promotes the release of origin recognition complex (ORC) proteins from chromatin when added to Xenopus egg extract or as purified components. Cdc2-cyclin A is combined with permeable donor nuclei in time course studies. The loss of bovine ORC proteins from chromatin is monitored by western blot. Donor cell nuclei are treated with nucleoplasmin (NPL), cyclin A-dependent protein kinase, and a combination of nucleoplasmin and cyclin A-dependent kinase. The resulting remodeled somatic nuclei are used for nuclear transfer.

Donor cells are permeabilized by homogenization in a tight-fitting dounce apparatus containing hypotonic buffer. The nuclei are then treated with nucleoplasmin, Cdc2/Cdk2-cyclin A, or nucleoplasmin and Cdc2/Cdk2-cyclin A, prior to nuclear transfer. A control group consists of an equal volume of buffer used to prepare the protein samples.

While these Examples illustrate the donor cell or nuclei being contacted with the remodeling factors prior to nuclear transfer, successful results may also be obtained by contacting the donor cell or nuclei with remodeling factors subsequent to or simultaneous with nuclear transfer.

### EXAMPLE 4

### Use of S-Phase Extracts from Activated Xenopus Eggs

Permeable cells (intact nuclei) are exposed to cytoplasmic extracts derived from activated Xenopus eggs arrested in S-phase of the cell cycle. This may facilitate the reorganization of chromatin in the absence of DNA replication. While the major changes in chromatin structure that occur during S-phase are associated with DNA synthesis, more subtle replication-independent processes may also be important for reprogramming. Confluent donor nuclei generally possess the origin recognition complex (ORC) proteins but generally do not possess Cdc6 or the minichromosome maintenance (MCM) proteins, all of which facilitate the initiation of DNA replication in eukaryotic cells. The Cdc6 and MCM proteins may be present in S-phase egg extract but are generally unable to bind chromatin surrounded by an intact nuclear envelope. Therefore, in addition to concentrating nuclear proteins, an intact envelope may prevent replication in S-phase extracts by preventing the assembly of pre-replication complexes on DNA. Pre-replication complexes may eventually assemble on donor cell DNA when the nuclear envelope breaks down in the recipient bovine egg.

While this example utilized extracts obtained from activated Xenopus eggs by way of example, other cell extracts, such as unactivated Xenopus eggs may also be utililized.

### EXAMPLE 5

### G2-Phase/Prophase Extracts from Xenopus Oocytes

Permeable cells (intact nuclei) are exposed to cytoplasmic extracts derived from late-stage Xenopus oocytes arrested in G2-phase of meiosis I. Late-stage oocytes are capable of transcription but not DNA replication, precisely the opposite of S-phase extracts from activated eggs. Without wanting to be bound by any particular theory, it is believed that oocyte extracts may alter somatic nuclei in at least two unique ways. First, they may modify nuclear structure and/or function to reflect the pre-mitotic or very early mitotic environment, possibly by facilitating increased chromosome condensation; and second, oocyte extracts may facilitate reprogramming by supplying transcription factors that are inactive or absent from bovine eggs.

### EXAMPLE 6

### Meiotic Metaphase Extracts from Metaphase-Arrested Xenopus Eggs

Permeable cells (intact nuclei) are exposed to extracts derived from metaphase-arrested Xenopus eggs. Somatic nuclei undergo nuclear envelope breakdown and chromosome condensation upon entering the bovine egg. These changes are the result of active cdc2-cyclin B, a protein kinase that promotes metaphase arrest. Without wanting to be bound by any particular theory, it is believed that these structural changes may facilitate the reprogramming of somatic DNA. It is also believed that release of the egg from metaphase arrest or “activation” may lead to the assembly of a diploid “pronucleus” and entry into the first mitotic cell cycle. Xenopus eggs are also arrested in metaphase of meiosis II and extracts from these eggs may mimic precisely the activities within mammalian eggs prior to activation. Mammalian somatic nuclei, incubated in these extracts, may undergo nuclear envelope breakdown and chromosome condensation. The resultant metaphase chromosomes may resemble those that are formed within the egg in the absence of activation. These metaphase chromosomes may be microinjected into the bovine egg directly or alternatively; they may be assembled into pronuclei by activating the metaphase-arrested egg extract with calcium. Exogenous calcium releases the extract from metaphase arrest in part by destabilizing Cdc2-cyclin B. The effects of experimental manipulations are determined by monitoring embryo development as described in Example 1.

### EXAMPLE 7

### Isolation and Culture of Genital Ridge Cells

Genital ridges were aseptically removed from bovine fetuses of age 40-80 days. The genital ridges were minced with surgical blades in 1 ml of Tyrodes Lactate Hepes (TL-Hepes) medium (Biowhittaker, Inc., Walkersville, Md., USA) containing protease from Streptomyces griseus (Sigma, St. Louis, Mo., USA, cat. # P6991) (3 mg/ml) and incubated at 37° C. for 45 min. The minced genital ridges were disaggregated by passing them through a 25-gauge needle several times. The disaggregated genital ridges were diluted with 10 ml of TL-Hepes medium and centrifuged at 300×g for 10 min. A portion of the pellet corresponding to 50,000-100,000 cells was cultured in Amniomax medium. All cultured cells were kept in an atmosphere of humidified air/5% CO₂ at 37° C. Upon reaching confluence, the cells were passaged using standard procedures.

### EXAMPLE 8

### Isolation and Culture of Cells from Fetal Body Tissue

Fetal bovine tissue corresponding to the outer part of the upper body minus the head and viscera was minced with scalpel blades and then digested in 5 ml of a trypsin-EDTA phosphate-buffered saline (Gibco, Rockville, Md., USA) solution for 45 minutes at 37° C. The digest was filtered through a: 70 μm mesh cell strainer and the effluent was centrifuged at 300×g for 10 min. A portion of the pellet corresponding to 50,000-100,000 cells was cultured in 35-mm culture dishes in α-MEM containing 0.1 mM 2-mercaptoethanol, 4 mM L-glutamine, and 10% FBS. The cells were passaged upon confluence. Fibroblast-like cells dominated most cultures of fetal body cells. However, fetal body cell cultures occasionally became dominated with cells that resembled epithelial-like GR cells cultured on mouse feeder layers.

### EXAMPLE 9

### Isolation and Culture of Cells from Bovine Ear Tissue

Small portions of the ear were aseptically removed and washed several times in phosphate buffered saline (PBS). The ear samples were minced with scalpel blades and then digested in 5 ml of a trypsin-EDTA phosphate-buffered saline solution for 45 minutes at 37° C. The digest was filtered through a 70 μm mesh cell strainer and the effluent was centrifuged at 300×g for 10 min. The pellet was resuspended and cultured in 35-mm culture dishes in A-MEM containing 0.1 mM 2-mercaptoethanol, 4 mM L-glutamine, and 10% fetal bovine serum. The cells were passaged upon confluence.

### EXAMPLE 10

### Isolation and Culture of Cumulus Cells

Oocytes aspirated from ovaries were matured overnight in maturation medium (Medium 199, Gibco) supplemented with luteinizing hormone (10 IU/ml, Sigma), estradiol (1 mg/ml, Sigma) and FBS (10%, Hyclone) at 38.5° C. in a humidified 5% CO₂ incubator. The oocytes were stripped of cumulus cells after 16-18 hours post onset of maturation by vortexing in 0.5 ml of TL-Hepes. The cumulus cells were collected and grown in α-MEM (Gibco) containing 0.1 mM 2-mercaptoethanol, 4 mM L-glutamine, and 10% fetal bovine serum. The cells were passaged upon confluence.

### EXAMPLE 11

### Remodelling of Bovine Cells

Oocytes aspirated from abattoir ovaries were matured overnight in maturation medium (Medium 199, Gibco) supplemented with luteinizing hormone (10 IU/ml, Sigma), estradiol (1 mg/ml, Sigma) and FBS (10%, Hyclone) at 38.5° C. in a humidified 5% CO₂ incubator. The oocytes were stripped of cumulus cells after 16-18 hours post onset of maturation by vortexing in 0.5 ml of TL-Hepes. The chromatin was stained with Hoechst 33342 (5 μg/ml, Sigma) in TL-Hepes solution. Stained oocytes were enucleated in drops of TL-Hepes solution under mineral oil. Cells used in the NT procedure were prepared by releasing confluent cells from a 13 nm diameter culture well by incubating in α-MEM (Gibco) containing 3 mg/ml S. griseus protease (Sigma) in 5% CO₂ incubator for the amount of time required to achieve single cell suspension (5-30 min). Once the cells were in a single cell suspension they were washed with TL-Hepes and used for NT within 2-3 hours. Single nuclear donor cells were inserted into the perivitelline space of the enucleated oocyte. The cell and oocyte plasma membranes were fused by applying an electrical pulse of 104V for 15 in an isotonic sorbitol solution (0.25 M) containing magnesium acetate (0.5 mM), and fatty acid free bovine serum albumin (BSA) (1 mg/ml, Sigma #A7030) (pH 7.2) but lacking calcium at 30° C. in a 500 μm fusion chamber. Following 4 hr of culture in CR1aa (CR2) medium [39] containing 3 mg/ml BSA, the NT embryos were activated by a 4 min exposure to 5 μM ionomycin (Ca²⁺-salt) (Sigma) in Hepes buffered TC199 containing 1 mg/ml BSA, followed by a 5 min wash TL-Hepes. The activated embryos were then incubated in CR2 medium containing 1.9 mM 6-dimethylaminopurine (DMAP, Sigma) for 3-5 hrs followed by a wash in TL-Hepes and subsequently cultured in CR2 medium with BSA (3 mg/ml) at 38.5° C. in a humidified 5% CO₂ incubator for four days. The embryos were transferred to CR2 medium containing 10% FBS and cultured for an additional 1-4 days.

Injection of Remodeling Factors

The NT-injection manipulation plate contained a small drop (10 μl) of remodeling factor to be injected. An injection tip approximately 8 μm in diameter at the orifice was placed into the drop containing remodeling factor and negative pressure was applied. After approximately two minutes of front loading the injection tip, positive pressure was exerted to carefully allow a weak flow of remodeling factor out of the tip. The tip was inserted through the hole created from enucleation and cell transfer. A single pulse from the PiezoDrill (2 Hz, 75 μS, 20 V) allowed the tip into the cytoplasm and approximately 300 pl was allowed to flow into the oocyte before the tip was withdrawn. Three different concentrations of nucleoplasmin (NPL) and four concentrations of polyglutamic acid (PGA, MW 13,600 Sigma # P-4636) were injected into oocytes within one-hour pre- or post-fusion of the donor cell using a PiezoDrill (Burleigh Instruments, Fishers, N.Y.). NPL was injected to an estimated final concentration of 100 ng/μl (300 ng/μl stock solution injected), 500 ng/μl (1500 ng/μl stock solution), or 2500 ng/μl (7500 ng/μl stock solution). The concentration of NPL in the Xenopus egg is approximately 500 ng/μl. Mills et al., J. Mol. Biol. 139: 561-8 (1980). Four separate NPL preparations (NPL2, NPL3, NPL4, and NPL5) and one mixed NPL preparation (NPLx) were used in the study. PGA was injected to an estimated final concentration of 100 ng/μl (300 ng/μl stock solution injected), 500 ng/μl (1500 ng/μl stock solution), 1000 ng/μl (3000 ng/l stock solution), or 2500 ng/μl (7500 ng/μl stock solution).

Embryo Transfer

Grade 1 or 2 blastocysts were used for transfer into recipients (one or two embryos/recipient). Recipients were observed for natural estrus and blastocysts were transferred into recipients whose predicted ovulation had occurred within 60 hours of the time that the nuclear donor cells were fused into the enucleated oocytes. Transfers occurred 6-8 days post fusion.

Results

Injection of Nucleoplasmin (NPL)

Four primary variables were components of this study: 1) donor cell line; 2) concentration of NPL injected; 3) method of NPL injection; and 4) NPL preparation injected.

1. Six cell lines were used in this study. Four of these lines were identified internally as adult fibroblast lines, one line as an adult cumulus line and one as a fetal EG line.

2. Three concentrations of NPL were injected into the bovine oocyte. Our goal was to inject NPL to an estimated final concentration of 500 ng/μl mimicking the concentration in the frog egg. We also injected NPL to an estimated final concentration of 100 ng/μl, representing a five-fold lower concentration of NPL, and to an estimated final concentration of 2500 ng/μl representing a five-fold higher concentration of NPL than that reported in the frog egg.

3. NPL was injected into the oocyte following donor cell fusion (method 1.5) or before donor cell fusion (method 1.6). The two methods differ in the time they provide NPL to form complexes with bovine cytoplasmic proteins before nuclear remodeling occurs in the bovine oocyte. NPL is bound to histones H2A.X and H2B in the frog oocyte and egg and assembles these proteins on sperm chromatin in Xenopus egg extracts.

4. Four individual preparations (NPL2, NPL3, NPL4, and NPL5) and one mixed NPL preparation (NPLx) were used in the study. The preliminary work, in which the injection technology was developed, was done with the mixed preparation (NPLx).

5. The data shown (Table 1) reflect only those NT days in which ETs were conducted. In other words, NTs that did not produce blastocysts or NTs in which blastocysts were produced but were not transferred, were not used to calculate the final numbers for blastocyst development. The values for 7500 ng/μl, 1.5, NPL3 and NPL5 are shown in Table 1, but are not included in the NPL totals. These values are included simply to demonstrate that injections were conducted within these groups.

6. NTs with injection buffer alone or NTs without NPL or buffer injection (Control) were conducted. The rate of blastocyst development in injection buffer controls (27.4%) was very similar to blastocyst development in NPL (21.6%) and in no injection controls (24.1%). Therefore, injection control embryos were not used for ETs in this study. Control (no injection) NTs were conducted over the same time period that the experimental NTs were done (Control-Same Time Matched). The study results are outlined in Table 2.

Summary

Similar rates of blastocyst development were observed between Control NT group and the Total NPL NT group and among the different NPL NT subgroups (i.e., groups of different concentration, method, or preparation)., The rate of pregnancy initiation between Control NT and total NPL NT groups was also similar. However, a wide variation in pregnancy initiation was observed among the NPL NT subgroups. The highest concentration of NPL (7500 ng/μl) produced the lowest level of pregnancy initiation (17.6%) while the highest rate of initiation (38.5%) occurred at the lowest concentration (300 ng/μl). A pregnancy initiation rate of 41.7% was observed at 1500 ng/μl using method 1.5, greater than the Control group (28.4%). Moreover, a pregnancy initiation rate of 71.4% (5/7) was observed with NPL preparation 3 within this group, the highest rate observed among all groups. One pregnancy in the 1500 ng/μl-1.5-NPL3 group also resulted in the birth of live, healthy calf.

TABLE 2
Injection of Nucleoplasmin (NPL) into Bovine Oocytes Before (1.6) or After
(1.5) Donor Cell Fusion

| Concentration | | | | | | | | | | | % Preg. | |
|---|---|---|---|---|---|---|---|---|---|---|---|---|
| Injected | Method | Prep | NTs | Blasts | (%) | ETs | ABORT (days) | HYDROPS | TERMIN | CALVED | Initiation | |
| 300 ng/μl | 1.5 | NPL2 | 19 | 4 | 21.1 | 2 | | | | | 0 | (0/2) |
| | | NPL3 | 20 | 5 | 25 | 1 | | | | | 0 | (0/1) |
| | | NPL4 | 14 | 2 | 14.3 | 1 | 1 (34) | | | | 100 | (1/1) |
| | | NPL5 | 35 | 3 | 8.6 | 2 | 1 (55) | | | | 50 | (1/2) |
| | Total | | 88 | 14 | 15.9 | 6 | 2 | | | | 33 | (2/6) |
| | 1.6 | NPL2 | 52 | 8 | 15.4 | 2 | 1 (41) | | | | 50 | (1/2) |
| | | NPL3 | 15 | 2 | 13.3 | 1 | 1 (38) | | | | 100 | (1/1) |
| | | NPL4 | 22 | 4 | 18.2 | 2 | | | 1 (99) | | 50 | (1/2) |
| | | NPL5 | 32 | 6 | 18.8 | 2 | | | | | 0 | (0/2) |
| | Total | | 121 | 20 | 16.5 | 7 | 2 | | 1 | | 42.9 | (3/7) |
| TOTAL | | | 209 | 34 | 16.3 | 13 | 4 | | 1 | | 38.5 | (5/13) |
| 1500 ng/μl | 1.5 | NPLx | 145 | 48 | 33.1 | 11 | 3 (46, 40, 31) | | | | 27.3 | (3/11) |
| | | NPL2 | 50 | 11 | 22 | 6 | 2 (151, 45) | 1 (279) | | | 50 | (3/6) |
| | | NPL3 | 72 | 15 | 20.8 | 7 | 2 (116, 41) | 1 (227) | 1 (61) | 1 (290) | 71.4 | (5/7) |
| | | NPL4 | 123 | 22 | 17.9 | 10 | 4 (62, 60, 34, 27) | | | | 40 | (4/10) |
| | | NPL5 | 27 | 3 | 11.1 | 2 | | | | | 0 | (0/2) |
| | Total | | 417 | 99 | 23.7 | 36 | 11 | 2 | 1 | 1 | 41.7 | (15/36) |
| | 1.6 | NPL2 | 88 | 24 | 27.3 | 7 | | 1 (232) | | | 14.3 | (1/7) |
| | | NPL3 | 38 | 7 | 18.4 | 4 | 2 (108, 46) | | | | 50 | (2/4) |
| | | NPL4 | 24 | 3 | 12.5 | 2 | | | | | 0 | (0/2) |
| | | NPL5 | 33 | 2 | 6.1 | 1 | | | | | 0 | (0/1) |
| | Total | | 183 | 36 | 19.7 | 14 | 2 | 1 | | | 21.4 | (3/14) |
| TOTAL | | | 600 | 135 | 22.5 | 50 | 13 | 3 | 1 | 1 | 36 | (18/50) |
| 7500 ng/μl | 1.5 | NPL2 | 25 | 16 | 64 | 7 | 2 (41, 34) | | | | 28.6 | (2/7) |
| | | NPL3* | 16 | 1 | 6.3 | 0 | | | | | | |
| | | NPL4 | 25 | 6 | 24 | 1 | | | | | 0 | (0/1) |
| | | NPL5* | 27 | 0 | 0 | 0 | | | | | | |
| | | Total | 50 | 22 | 44 | 8 | 2 | | | | 25 | (2/8) |
| | 1.6 | NPL2 | 32 | 7 | 21.9 | 3 | 1 (41) | | | | 33.3 | (1/3) |
| | | NPL3 | 26 | 8 | 30.8 | 4 | | | | | 0 | (0/4) |
| | | NPL4 | 23 | 2 | 8.7 | 1 | | | | | 0 | (0/1) |
| | | NPL5 | 31 | 2 | 9.5 | 1 | | | | | 0 | (0/1) |
| | | Total | 112 | 19 | 17 | 9 | 1 | | | | 11.1 | (1/9) |
| TOTAL | | | 162 | 41 | 25.3 | 17 | 3 | | | | 17.6 | (3/17) |
| NPL-Total | | | 971 | 210 | 21.6 | 80 | 20 | 3 | 2 | 1 | 32.5 | (26/80) |
| Control-Same Time | | | 1821 | 439 | 24.1 | 102 | 24 | 1 | | 4 | 28.4 | (29/102) |

*Values not included in Totals

Injection of Polyglutamic Acid (PGA)

Three primary variables were components of this study: 1) donor cell line; 2) concentration of PGA injected; and 3) method of PGA injection.

1. Four cell lines were used in this study. Three of the four lines used (2 adult fibroblast and 1 adult cumulus) were the same lines used in the NPL study. The remaining line was a fetal EG line different from the fetal EG line used in the NPL study.

2. Four concentrations of PGA were injected into the bovine oocyte: We injected PGA to an estimated final concentration of 100 ng/μl (300 ng/μl stock), 500 ng/μl (1500 ng/μl stock), 1000 ng/μl (3000 ng/μl stock), and 2500 ng/μl (7500 ng/μl stock).

3. PGA was injected into the oocyte following donor cell fusion (method 1.5) or before donor cell fusion (method 1.6). The two methods differ in the time they provide PGA to form complexes with bovine cytoplasmic proteins before nuclear remodeling occurs in the bovine oocyte. PGA has been shown to assemble histones on sperm DNA forming nucleosomes. Dean, Dev. Biol. 99: 210-6 (1983).

4. The data shown (Table 3) reflect only those NT days in which ETs were conducted. In other words, NTs that did not produce blastocysts or NTs in which blastocysts were produced but were not transferred, were not included in the results.

NTs without PGA injection were conducted (no injection Controls). All control NTs were performed on the same cell lines that were used for PGA injection. Control NTs are those that were conducted over the same time period that the PGA NTs were done (Control-Same Time Matched). The study results are outlined in Table 3.

TABLE 3
Injection of Polyglutamic Acid (PGA) into Bovine Oocytes Before (1.6) or
After (1.5) Donor Cell Fusion

| Concentration | | | | | | | ABORT | | | | % Pregnancy | |
|---|---|---|---|---|---|---|---|---|---|---|---|---|
| Injected | Method | Prep | NTs | Blasts | (%) | ETs | (days) | HYDROPS | TERMIN | CALVED | Initiation | |
| 300 ng/μl | 1.5 | PGA | 50 | 12 | 24 | 3 | | | | | 0 | (0/3) |
| | 1.6 | PGA | 54 | 14 | 25.9 | 3 | 1 (41) | | | | 33.3 | (1/3) |
| TOTAL | | | 104 | 26 | 25 | 6 | 1 | | | | 16.6 | (1/6) |
| 1500 ng/μl | 1.5 | PGA | 50 | 13 | 26 | 4 | 1 (41) | | | | 25 | (1/4) |
| | 1.6 | PGA | 57 | 12 | 21.1 | 3 | 1 (41) | | | | 33.3 | (1/3) |
| TOTAL | | | 107 | 25 | 23.4 | 7 | 2 | | | | 28.6 | (2/7) |
| 3000 ng/μl | 1.5 | PGA | 34 | 6 | 17.6 | 2 | | | | | 0 | (0/2) |
| | 1.6 | PGA | 56 | 14 | 25 | 3 | 3 (31, 32, 81) | | | | 100 | (3/3) |
| TOTAL | | | 90 | 20 | 22.2 | 5 | 3 | | | | 60 | (3/5) |
| 7500 ng/μl | 1.5 | PGA | 32 | 2 | 6.3 | 2 | | | | | 0 | (0/2) |
| | 1.6 | PGA | 53 | 9 | 17 | 2 | 1 (41) | | | | 50 | (1/2) |
| TOTAL | | | 85 | 11 | 12.9 | 4 | 1 | | | | 25 | (1/4) |
| TOTAL PGA | | | 386 | 82 | 21.2 | 22 | 7 | | | | 31.8 | (7/22) |
| Control-Same | | | 916 | 213 | 23.3 | 24 | 3 | | | | 12.5 | (3/24) |
| Time | | | | | | | | | | | | |

Summary

Similar rates of blastocyst development were observed between the Control NT group and the Total PGA NT group and among the different PGA NT subgroups (i.e., groups of different concentration and method). Similar to NPL, the highest concentration of PGA had the lowest rate of blastocyst development (12.9%). However, comparing the rate of pregnancy initiation between Total PGA NT and the Control NT groups revealed that the Total PGA rate (31.8%) was higher that the Control group rate (12.5%). Furthermore, one PGA NT subgroup (3000 ng/μl-1.6) had an initiation rate of 100% (3/3).

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Other embodiments are set forth within the following claims.

1. A method for preparing a mammalian embryo by nuclear transfer, comprising:
(a) transferring a mammalian cell, or the nucleus thereof, into an enucleated mammalian NT oocyte;
(b) introducing into the mammalian NT oocyte one or more remodeling factors prior to, subsequent to, or simultaneous with said transferring step (a); and
(c) activating said mammalian NT oocyte to provide said embryo.

2. A method for cloning a mammal by nuclear transfer, comprising:
(a) preparing an embryo by the method of claim 1; and
(b) transferring the embryo or a re-cloned embryo thereof into the uterus of a host mammal so as to produce a fetus that undergoes full development and parturition.

3. The method of claim 1 or 2, wherein the remodeling factors are obtained from cells selected from the group consisting of Xenopus oocytes, Xenopus eggs, and activated Xenopus eggs.

4. The method of claim 1 or 2, wherein the mammalian NT oocyte is a bovine egg, and the mammalian cell is a bovine cell.

5. The method of claim 1 or 2, wherein the mammalian NT oocyte is a porcine egg, and the mammalian cell is a porcine cell.

6. The method of claim 1 or 2, wherein the mammalian NT oocyte is an ovine egg, and the mammalian cell is an ovine cell.

7. The method of claim 1, wherein the step of introducing said one or more remodeling factors into the mammalian NT oocyte occurs subsequent to said transferring step (a).

8. The method of claim 1, wherein said transferring step (a) comprises fusing the mammalian cell and the egg.

9. The method of claim 1 or 2, wherein the one or more remodeling factors are introduced into the egg by microinjection.

10. The method of claim 1 or 2, wherein one of said one or more remodeling factor(s) is nucleoplasmin.

11. The method of claim 1 or 2, wherein one of said one or more remodeling factor(s) is a cyclin A-dependent kinase.

12. The method of claim 1 or 2, wherein said one or more remodeling factor(s) comprise cyclin A-dependent kinase and nucleoplasmin.

13. The method of claim 1 or 2, wherein the mammalian cell is selected from the group consisting of: an embryonic cell, a fetal cell, a fetal fibroblast cell, an adult cell, a somatic cell, a primordial germ cell, a genital ridge cell, a fibroblast cell, a cumulus cell, an amniotic cell, an embryonic germ cell, an embryonic stem cell, an ovarian follicular cell, a hepatic cell, an epidermal cell, an epithelial cell, a hematopoietic cell, keratinocyte, a renal cell, a lymphocyte, a melanocyte, a muscle cell, a myeloid cell, a neuronal cell, an osteoblast, a mysenchymal cell, a mesodermal cell, an adherent cell, a cell isolated from an asynchronous population of cells, a cell isolated from a synchronous population of cells where the synchronous population is not arrested in the G₀ stage of the cell cycle, a cell isolated from a confluent culture, a transgenic embryonic cell, a transgenic fetal cell, a transgenic adult cell, a transgenic somatic cell, a transgenic primordial germ cell, a transgenic fibroblast cell, a transgenic cumulus cell, or a transgenic amniotic cell.

14. A method for preparing a mammalian embryo by nuclear transfer, comprising:
(a) transferring a mammalian cell, or the nucleus thereof, into an enucleated mammalian NT oocyte;
(b) introducing into the mammalian NT oocyte a cytoplasmic extract obtained from one or more cells selected from the group consisting of Xenopus oocytes, Xenopus eggs, and activated Xenopus eggs, prior to, subsequent to, or simultaneous with said transferring step (a); and
(c) activating said mammalian NT oocyte to provide said embryo.

15. A method for cloning a mammal, comprising:
(a) preparing an embryo by the method of claim 14; and
(b) transferring the embryo or a re-cloned embryo thereof into the uterus of a host mammal so as to produce a fetus that undergoes full development and parturition.

16. The method of claim 14 or 15, wherein the mammalian NT oocyte is a bovine egg, and the mammalian cell is a bovine cell.

17. The method of claim 14 or 15, wherein the mammalian NT oocyte is a porcine egg, and the mammalian cell is a porcine cell.

18. The method of claim 14 or 15, wherein the mammalian NT oocyte is an ovine egg, and the mammalian cell is an ovine cell.

19. The method of claim 14 or 15, wherein the mammalian cell is selected from the group consisting of: an embryonic cell, a fetal cell, a fetal fibroblast cell, an adult cell, a somatic cell, a primordial germ cell, a genital ridge cell, a fibroblast cell, a cumulus cell, an amniotic cell, an embryonic germ cell, an embryonic stem cell, an ovarian follicular cell, a hepatic cell, an epidermal cell, an epithelial cell, a hematopoietic cell, keratinocyte, a renal cell, a lymphocyte, a melanocyte, a muscle cell, a myeloid cell, a neuronal cell, an osteoblast, a mysenchymal cell, a mesodermal cell, an adherent cell, a cell isolated from an asynchronous population of cells, a cell isolated from a synchronous population of cells where the synchronous population is not arrested in the G₀ stage of the cell cycle, a cell isolated from a confluent culture, a transgenic embryonic cell, a transgenic fetal cell, a transgenic adult cell, a transgenic somatic cell, a transgenic primordial germ cell, a transgenic fibroblast cell, a transgenic cumulus cell, or a transgenic amniotic cell.

20. A method for preparing a mammalian embryo by nuclear transfer, comprising:
(a) contacting a mammalian cell, or a nucleus thereof, with one or more remodeling factors;
(b) transferring the mammalian cell, or the nucleus thereof, into an enucleated mammalian NT oocyte; and
(c) activating said egg to provide said embryo.

21. A method for cloning a mammal by nuclear transfer, comprising:
(a) preparing an embryo by the method of claim 20; and
(b) transferring the embryo or a re-cloned embryo thereof into the uterus of a host mammal so as to produce a fetus that undergoes full development and parturition.

22. The method of claim 20 or 21, wherein the remodeling factors are obtained from cells selected from the group consisting of Xenopus oocytes, Xenopus eggs, and activated Xenopus eggs.

23. The method of claim 20 or 21, wherein the plasma membrane of the mammalian cell is permeabilized.

24. The method of claim 20 or 21, wherein the nuclear membrane of the mammalian cell nucleus is permeabilized.

25. The method of claim 23, wherein the plasma membrane of the mammalian cell is permeabilized by exposure to streptolysin-O and/or digitonin prior to contacting the mammalian cell with one or more remodeling factors.

26. The method of claim 20 or 21, wherein the remodeling factors are nucleoplasmin and/or protein kinases.

27. The method of claim 26 wherein the protein kinase is Cdc2, Cdk2, or a combination thereof.

28. The method of claim 20 or 21, wherein the mammalian NT oocyte is a bovine egg, and the mammalian cell is a bovine cell.

29. The method of claim 20 or 21, wherein the mammalian NT oocyte is a porcine egg, and the mammalian cell is a porcine cell.

30. The method of claim 20 or 21, wherein the mammalian NT oocyte is an ovine egg, and the mammalian cell is an ovine cell.

31. The method of claim 20 or 21, wherein the mammalian cell is selected from the group consisting: an embryonic cell, a fetal cell, a fetal fibroblast cell, an adult cell, a somatic cell, a primordial germ cell, a genital ridge cell, a fibroblast cell, a cumulus cell, an amniotic cell, an embryonic germ cell, an embryonic stem cell, an ovarian follicular cell, a hepatic cell, an epidermal cell, an epithelial cell, a hematopoietic cell, keratinocyte, a renal cell, a lymphocyte, a melanocyte, a muscle cell, a myeloid cell, a neuronal cell, an osteoblast, a mysenchymal cell, a mesodermal cell, an adherent cell, a cell isolated from an asynchronous population of cells, a cell isolated from a synchronous population of cells where the synchronous population is not arrested in the G0 stage of the cell cycle, a transgenic embryonic cell, a transgenic fetal cell, a transgenic adult cell, a transgenic somatic cell, a transgenic primordial germ cell, a transgenic fibroblast cell, a transgenic cumulus cell, or a transgenic amniotic cell.

32. A method for preparing a mammalian embryo by nuclear transfer, comprising:
(a) contacting a mammalian cell, or a nucleus thereof, with a cytoplasmic extract obtained from one or more cells selected from the group consisting of Xenopus oocytes, Xenopus eggs, and activated Xenopus eggs;
(b) transferring the mammalian cell, or the nucleus thereof, into an enucleated mammalian NT oocyte; and
(c) activating said mammalian NT oocyte to provide said embryo.

33. A method for cloning a mammal, comprising:
(a) preparing an embryo by the method of claim 32; and
(b) transferring the embryo or a re-cloned embryo thereof into the uterus of a host mammal so as to produce a fetus that undergoes full development and parturition.

34. The method of claim 32 or 33, wherein the plasma membrane of the mammalian cell is permeabilized by exposure to streptolysin-O and/or digitonin.

35. The method of claim 32 or 33, wherein the nuclear membrane of the mammalian cell nucleus is permeabilized.

36. The method of claim 35, wherein the nuclear membrane of the mammalian cell nucleus is permeabilized by homogenization.

37. The method of claim 32 or 33, wherein the mammalian cell is selected from the group consisting of: an embryonic cell, a fetal cell, a fetal fibroblast cell, an adult cell, a somatic cell, a primordial germ cell, a genital ridge cell, a fibroblast cell, a cumulus cell, an amniotic cell, an embryonic germ cell, an embryonic stem cell, an ovarian follicular cell, a hepatic cell, an epidermal cell, an epithelial cell, a hematopoietic cell, keratinocyte, a renal cell, a lymphocyte, a melanocyte, a muscle cell, a myeloid cell, a neuronal cell, an osteoblast, a mysenchymal cell, a mesodermal cell, an adherent cell, a cell isolated from an asynchronous population of cells, a cell isolated from a synchronous population of cells where the synchronous population is not arrested in the G0 stage of the cell cycle, a transgenic embryonic cell, a transgenic fetal cell, a transgenic adult cell, a transgenic somatic cell, a transgenic primordial germ cell, a transgenic fibroblast cell, a transgenic cumulus cell, or a transgenic amniotic cell.
